(19) United States
(12) Patent Application Publication
(10) Pub. No.: US 2026/0027062 A1
(43) Pub. Date: Jan. 29, 2026

(54) IMPROVED ORAL PHARMACEUTICAL FORMULATIONS OF THERAPEUTIC PEPTIDES AND PROTEINS

(71) Applicant: CYPRUMED GMBH, Obsteig (AT)

(72) Inventors: Florian FÖGER, Obsteig (AT); Martin WERLE, Obsteig (AT)

(73) Assignee: CYPRUMED GMBH, Obsteig (AT)

(21) Appl. No.: 18/840,653

(22) PCT Filed: Mar. 3, 2023
(86) PCT No.: PCT/EP2023/055446

§ 371 Date: Aug. 22, 2024

(30) Foreign Application Priority Data
Mar. 3, 2022 (EP) 22159924.4

(51) Int. Cl. A61K 9/48; A61K 9/00; A61K 9/28; A61K 38/09; A61K 38/26; A61K 38/28; A61K 38/31; A61K 45/00; A61K 47/32

(52) U.S. Cl. CPC A61K 9/4891; A61K 9/0053; A61K 9/2846; A61K 9/2886; A61K 38/09; A61K 38/26; A61K 38/28; A61K 38/31; A61K 45/00; A61K 47/32

(57) ABSTRACT

The present invention relates to a solid oral pharmaceutical composition comprising (i) a core comprising a peptide or protein drug, and (ii) a first coating, wherein the first coating comprises a copolymer (A) in combination with a copolymer (B) and/or a copolymer (C) and/or a copolymer (D).

The present invention relates to a solid oral pharmaceutical composition comprising (i) a core comprising a peptide or protein drug, and (ii) a first coating, wherein the first coating comprises a copolymer (A) in combination with a copolymer (B) and/or a copolymer (C) and/or a copolymer (D). The present application is a § 371 national phase of International Application No. PCT/EP2023/055446 filed Mar. 3, 2023, which claims the priority of European patent application EP 22 159 924.4 filed on Mar. 3, 2022, the contents of both of which are incorporated herein by reference in their entirety.

### REFERENCE TO A SEQUENCE LISTING

This application contains a Sequence Listing XML, which has been submitted electronically and is hereby incorporated by reference in its entirety. Said Sequence Listing XML, created on Mar. 3, 2025, is named VOSSP0144US—Sequence Listing.xml, and is 10,010 bytes in size.

A growing number of peptides and proteins have been made available as therapeutic agents. However, the full potential of these biological drugs has not been realized because they are typically limited to parenteral injection. Ideally, the oral route of administration would be preferred. Oral administration is the most common and popular method of administering drugs due to its simplicity and convenience for patients. However, the gastrointestinal tract degrades peptide and protein drugs and prevents their absorption as intact entities. Enzymatic degradation throughout the gastrointestinal tract and poor permeability through the epithelial cells are the main reasons for their low oral bioavailability.

A number of different approaches have been proposed to improve the oral bioavailability of such therapeutic peptides and proteins, including the use of absorption enhancing technologies or the use of protease inhibitors such as soybean trypsin inhibitor, aprotinin, bowman birk inhibitor, bacitracin, camostat mesilate and amastatin (Renukuntla J et al., Int J Pharm. 2013, 447(1-2):75-93; US 2007/0087957 A1). Due to safety concerns, however, none of these protease inhibitors have succeeded as additives in commercial peptide or polypeptide drug delivery applications.

Indeed, the development of oral dosage forms of therapeutic peptides and proteins that allow absorption to the systemic circulation has been described as one of the greatest challenges for the pharmaceutical industry (Aguirre, T A S et al., Adv Drug Deliv Rev. 2016, 106(Pt B), 223-241). Current enteric coating technologies suffer from several limitations such as liability to negative food interactions. Per-oral dosing of such dosage forms in presence of food results in significantly reduced oral bioavailability. Furthermore, Eudragit FS30D coated tablets with a peptide drug have been reported to result in several fold reduced oral bioavailability after storage (WO 2016/119854, FIG. 3). In addition, there are numerous reports about in vivo failure of enteric coated solid oral dosage forms (Hodson A H, J R Soc Med, 1994, 87(3):183; Grosser T et al., Circulation, 2013, 127(3):377-85). Especially enteric coatings which require a relatively high intestinal pH, such as above pH 7, are prone to failed dosing or incomplete dissolution most likely due to individuals' small intestinal pH variations, such as too short in vivo exposure to a sufficiently high pH.

Hence, there is still a pressing and unmet need for novel and improved pharmaceutical formulations of peptide or protein drugs, which are suitable for oral administration.

The present invention addresses these shortcomings in the art and provides solid oral pharmaceutical compositions of peptide or protein drugs, which are particularly well suited for oral administration due to their advantageous release profile, improved bioavailability and reduced food effects. A particular advantage of the current invention is that much lower amounts of pH dependent polymers are necessary due to combination with dominating time-dependent polymers. Such combinations with sufficiently high amounts of a time-dependent polymer result in surprisingly high oral bioavailability, reduced negative food impact, reduced variability and the avoidance of failed in vivo dissolution/disintegration. Further, these compositions are advantageously storage stable.

Accordingly, the present invention provides a solid oral pharmaceutical composition comprising:
(i) a core comprising a peptide or protein drug, and
(ii) a first coating, wherein the first coating comprises
   (ii-1) a copolymer (A) in combination with
   (ii-2) a copolymer (B) and/or a copolymer (C) and/or a copolymer (D);
   wherein the copolymer (A) comprises:
      (a) 20 to 90 mol-% ethyl acrylate repeating units, and
      (b) 10 to 80 mol-% methyl methacrylate repeating units;
   wherein the copolymer (B), if present, comprises:
      (a) 25 to 75 mol-% methacrylic acid repeating units, and
      (b) 25 to 75 mol-% ethyl acrylate repeating units;
   wherein the copolymer (C), if present, comprises:
      (a) 25 to 60 mol-% methacrylic acid repeating units, and
      (b) 40 to 75 mol-% methyl methacrylate repeating units;
   wherein the copolymer (D), if present, comprises:
      (a) 5 to 20 mol-% methacrylic acid repeating units, and
      (b) 20 to 40 mol-% methyl methacrylate repeating units, and
      (c) 60 to 75 mol-% methyl acrylate repeating units.

### FIGURES

FIG. 1: PK profile of semaglutide after oral dosing of Eudragit NM30D (80%)/Eudragit FS30D (20%) coated tablets in the fasted or fed state (-o- fasted group; -·-fed group, means of n=6±S.E.) (Example 9).

FIG. 2: In vitro stability of enteric dosage forms in simulated fed state gastric media as a function of release of octreotide (Example 18).

FIG. 3: In vitro stability of enteric dosage forms in simulated fed state gastric media as a function of release of leuprolide (Example 18).

FIG. 4: In vitro stability of enteric dosage forms in simulated fed state gastric media as a function of release of insulin (Example 18).

FIG. 5: Dissolution profile of octreotide (A), leuprolide (B) and insulin (C) from capsules coated with Eudragit L30D55 (reference) or a combination of 80% Eudragit NM30D and 20% Eudragit L30D55 (Example 19).

FIG. 6: Dissolution profile of tablets containing octreotide (black markers) and semaglutide (white markers) in SGF (1 hour) and SIF pH 7.4 (5 hours) with different coatings (L30D55 coating: circles, NM30D/FS30D coating: squares; n=3±S.D.) (Example 26).

In the context of the present invention, it was surprisingly found that the coating of the solid oral pharmaceutical composition provided herein results in an advantageous release profile significantly below pH 7 upon oral administration, allowing the release of the peptide or protein drug in the distal small intestine, as well as an improved bioavailability as compared to conventional formulations of peptide or protein drugs, including formulations with pH dependent enteric coatings. In particular, the pharmaceutical composition according to the invention has been found to allow dissolution at a comparatively low pH between 5.5 and 6.5. The release of the peptide or protein drug in the distal small intestine (distal jejunum or ileum) is advantageous in view of the reduced activity of proteolytic enzymes as compared to the proximal small intestine (duodenum and jejunum), in view of the reduced intestinal motility in this segment (leading to reduced dilution effects of the dissolving pharmaceutical composition, enabling a high concentration of the peptide or protein drug to achieve optimal absorption), and in view of the higher solubility of peptide or protein drugs at the pH levels present in the distal jejunum or ileum as compared to those present in the stomach, duodenum or proximal jejunum. In addition, it has been found that the solid oral pharmaceutical composition of the present invention exhibits advantageously reduced negative food effects. This stands in contrast to known formulations of various peptide or protein drugs that target the proximal gastrointestinal tract, in particular those targeting and dissolving in the proximal GI-tract such as the stomach or duodenum having no coating or a coating consisting only of anionic polymers, where detrimental food interactions have been observed (Maarbjerg S J et al., Diabetes, 2017, 66: A321 (without coating), as well as Example 33 of WO 2016/120378 A1 (coating based on Eudragit FS 30 D)). The solid oral pharmaceutical composition of the present invention can thus deliver the peptide or protein drug with an improved independence from food intake by the subject to be treated.

The present invention further provides a solid oral pharmaceutical composition (as described above) for use in therapy, particularly for use in the treatment or prevention of a disease/disorder (as detailed further below). It will be understood that the disease/disorder to be treated or prevented is a disease/disorder susceptible to treatment or prevention with the respective peptide or protein drug.

The invention likewise relates to the use of a solid oral pharmaceutical composition (as described above) for the manufacture of a medicament for the treatment or prevention of a disease/disorder.

Moreover, the invention refers to a method of treating or preventing a disease/disorder in a subject, the method comprising orally administering the solid oral pharmaceutical composition (as described above) to a subject in need thereof. It will be understood that a therapeutically effective amount should be administered in accordance with this method.

The invention also provides a method of orally delivering a peptide or protein drug, the method comprising orally administering the solid oral pharmaceutical composition (as described above).

It is preferred that the first coating dissolves at a pH in the range of 5 to 7, preferably at a pH in the range of 5.5 to 6.5, even more preferably at a pH in the range of 5.5 to 6.0. The components and optional components of the first coating, such as copolymers (A), (B), (C) and (D), will be described in the following.

### Copolymer (A)

Copolymer (A), which is present in the first coating, comprises (a) 20 to 90 mol-% ethyl acrylate repeating units, and (b) 10 to 80 mol-% methyl methacrylate repeating units. The copolymer (A) preferably is a neutral copolymer or a cationic copolymer.

It is particularly preferred that the copolymer (A) in the first coating is a neutral non-ionic copolymer. Preferably at least 90 mol-%, more preferably at least 95 mol-%, even more preferably at least 98 mol-%, of the repeating units in copolymer (A) are selected from ethyl acrylate repeating units and methyl methacrylate repeating units. In particular, the copolymer (A) may consist of ethyl acrylate repeating units and methyl methacrylate repeating units. The copolymer (A) in the first coating preferably comprises 50 to 80 mol-% ethyl acrylate repeating units and 20 to 50 mol-% methyl methacrylate repeating units, more preferably 60 to 75 mol-% ethyl acrylate repeating units and 25 to 40 mol-% methyl methacrylate repeating units, even more preferably 64 to 68 mol-% ethyl acrylate repeating units and 32 to 36 mol-% methyl methacrylate repeating units. The molar ratio of ethyl acrylate repeating units to methyl methacrylate repeating units in the copolymer (A) is preferably from 1.5:1 to 2.5:1, more preferably from 1.8:1 to 2.2:1, even more preferably 2:1. A corresponding preferred example of copolymer (A) is poly(ethyl acrylate-co-methyl methacrylate) 2:1, particularly Eudragit NM 30 D, Eudragit NE 30 D, or Eudragit NE 40 D.

As explained above, the copolymer (A) in the first coating may also be a cationic copolymer. Accordingly, the copolymer (A) in the first coating may further comprise 0.5 to 20 mol-%, preferably 1 to 15 mol-%, of 2-(trimethylammonio)ethyl methacrylate chloride repeating units (in addition to the ethyl acrylate repeating units and the methyl methacrylate repeating units). For example, in a preferred composition, the copolymer (A) in the first coating comprises 25 to 39 mol-% ethyl acrylate repeating units, 60 to 74 mol-% methyl methacrylate repeating units, and 1 to 15 mol-% 2-(trimethylammonio)ethyl methacrylate chloride repeating units. In the case that copolymer (A) is a cationic copolymer, it is preferred that at least 90 mol-%, more preferably at least 95 mol-%, even more preferably at least 98 mol-%, of the repeating units in copolymer (A) are selected from ethyl acrylate repeating units, methyl methacrylate repeating units, and 2-(trimethylammonio)ethyl methacrylate chloride repeating units. In particular, copolymer (A) in the first coating may consist of ethyl acrylate repeating units, methyl methacrylate repeating units, and 2-(trimethylammonio)ethyl methacrylate chloride repeating units. The copolymer (A) in the first coating may comprise ethyl acrylate repeating units, methyl methacrylate repeating units, and 2-(trimethylammonio)ethyl methacrylate chloride repeating units, e.g., in a molar ratio of 1:2:0.1 or 1:2:0.2. Corresponding preferred examples of copolymer (A) are poly(ethyl acrylate-co-methyl methacrylate-co-2-(trimethylammonio)ethyl methacrylate chloride) 1:2:0.2, particularly Eudragit RL 30 D, or poly(ethyl acrylate-co-methyl methacrylate-co-2-(trimethylammonio)ethyl methacrylate chloride) 1:2:0.1, particularly Eudragit RS 30 D.

It is generally preferred that the copolymer (A) in the first coating does not comprise methyl acrylate repeating units. Thus, the copolymer (A) in the first coating preferably comprises not more than 3 mol-% methyl acrylate repeating units, more preferably not more than 1 mol-%, even more preferably not more than 0.5 mol-%, yet more preferably not more than 0.1 mol-%, still more preferably not more than 0.01 mol-%, most preferably 0 mol-% methyl acrylate repeating units.

It is preferred that the copolymer (A) in the first coating is obtained from an aqueous dispersion of copolymer (A).

The first coating may further comprise, in addition to the copolymer (A), one or more polymers selected from ethyl cellulose, hydroxypropyl methylcellulose (HPMC), and polyvinyl acetate. Moreover, the present invention in a further embodiment also relates to a solid oral pharmaceutical composition as described and defined herein, wherein the first coating comprises one or more polymers selected from ethyl cellulose, hydroxypropyl methylcellulose (HPMC), and polyvinyl acetate instead of the copolymer (A).

### Copolymer (B)

The copolymer (B), if present in the first coating, comprises (a) 25 to 75 mol-% methacrylic acid repeating units and (b) 25 to 75 mol-% ethyl acrylate repeating units, preferably 45 to 55 mol-% methacrylic acid repeating units and 45 to 55 mol-% ethyl acrylate repeating units. The copolymer (B) preferably is an anionic copolymer. For example, the copolymer (B) in the first coating may comprise methacrylic acid repeating units and ethyl acrylate repeating units in a molar ratio of 0.5:1 to 1:0.5, preferably in a molar ratio of 0.8:1 to 1:08, more preferably in a molar ratio of 1:1. Preferably at least 90 mol-%, more preferably at least 95 mol-%, even more preferably at least 98 mol-%, of the repeating units in copolymer (B) are selected from methacrylic acid repeating units and ethyl acrylate repeating units. It is furthermore preferred that the copolymer (B) in the first coating consists of methacrylic acid repeating units and ethyl acrylate repeating units. A corresponding preferred example of copolymer (B) is poly(methacrylic acid-co-ethyl acrylate) 1:1, particularly Eudragit L 30 D-55 or Eudragit L 100 D-55.

It is generally preferred that the copolymer (B), if present in the first coating, does not comprise methyl acrylate repeating units. Thus, the copolymer (B) in the first coating preferably comprises not more than 3 mol-% methyl acrylate repeating units, more preferably not more than 1 mol-%, even more preferably not more than 0.5 mol-%, yet even more preferably not more than 0.1 mol-%, still more preferably not more than 0.01 mol-%, most preferably 0 mol-% methyl acrylate repeating units.

It is preferred that the copolymer (B) in the first coating is obtained from an aqueous dispersion of copolymer (B).

### Copolymer (C)

The copolymer (C), if present in the first coating, comprises (a) 25 to 60 mol-% methacrylic acid repeating units and (b) 40 to 75 mol-% methyl methacrylate repeating units. The copolymer (C) preferably is an anionic copolymer. Preferably at least 90 mol-%, more preferably at least 95 mol-%, even more preferably at least 98 mol-%, of the repeating units in copolymer (C) are selected from methacrylic acid repeating units and methyl methacrylate repeating units. In particular, the copolymer (C) in the first coating may consist of methacrylic acid repeating units and methyl methacrylate repeating units. The copolymer (C) in the first coating preferably is a copolymer (C-1) or a copolymer (C-2), as described in the following. Accordingly, the first coating may comprise a copolymer (C-1), a copolymer (C-2), or the combination of both a copolymer (C-1) and a copolymer (C-2).

The copolymer (C-1) in the first coating comprises 25 to 60 mol-% methacrylic acid repeating units and 40 to 75 mol-% methyl methacrylate repeating units, preferably 45 to 55 mol-% methacrylic acid repeating units and 45 to 55 mol-% methyl methacrylate repeating units. Preferably at least 90 mol-%, more preferably at least 95 mol-%, even more preferably at least 98 mol-%, of the repeating units in copolymer (C-1) are selected from methacrylic acid repeating units and methyl methacrylate repeating units. For example, the copolymer (C-1) in the first coating may comprise methacrylic acid repeating units and methyl methacrylate repeating units in a molar ratio of 0.5:1 to 1.5:1, preferably in a molar ratio of 0.8:1 to 1:08, more preferably in a molar ratio of 1:1. In particular, the copolymer (C-1) in the first coating may consist of methacrylic acid repeating units and methyl methacrylate repeating units. A corresponding preferred example of copolymer (C-1) is poly(methacrylic acid-co-methyl methacrylate) 1:1, particularly Eudragit L 100 or Eudragit L 12.5.

The copolymer (C-2) in the first coating comprises 25 to 60 mol-% methacrylic acid repeating units and 40 to 75 mol-% methyl methacrylate repeating units, preferably 25 to 40 mol-% methacrylic acid repeating units and 60 to 75 mol-% methyl methacrylate repeating units. Accordingly, it is preferred that the copolymer (C-2) in the first coating comprises methacrylic acid repeating units and methyl methacrylate repeating units in a molar ratio of 1:1.5 to 1:2.5, more preferably in a molar ratio of 1:2. Preferably at least 90 mol-%, more preferably at least 95 mol-%, even more preferably at least 98 mol-%, of the repeating units in copolymer (C-2) are selected from methacrylic acid repeating units and methyl methacrylate repeating units. In particular, the copolymer (C-2) in the first coating may consist of methacrylic acid repeating units and methyl methacrylate repeating units. A corresponding preferred example of copolymer (C-2) is poly(methacrylic acid-co-methyl methacrylate) 1:2, particularly Eudragit S 100.

It is generally preferred that the copolymer (C), if present in the first coating, including copolymer (C-1) and/or copolymer (C-2), does not comprise methyl acrylate repeating units. Thus, the copolymer (C) in the first coating (or the copolymer (C-1) and/or the copolymer (C-2)) preferably comprises not more than 3 mol-% methyl acrylate repeating units, more preferably not more than 1 mol-%, even more preferably not more than 0.5 mol-%, yet even more preferably not more than 0.1 mol-%, still more preferably not more than 0.01 mol-%, most preferably 0 mol-% methyl acrylate repeating units.

It is preferred that the copolymer (C) in the first coating, including the copolymer (C-1) and/or the copolymer (C-2), is obtained from an aqueous dispersion of the respective copolymer (i.e., copolymer (C), copolymer (C-1) or copolymer (C-2)).

### Copolymer (D)

The copolymer (D), if present in the first coating, comprises (a) 5 to 20 mol-% methacrylic acid repeating units, (b) 20 to 40 mol-% methyl methacrylate repeating units, and (c) 60 to 75 mol-% methyl acrylate repeating units. Preferably at least 90 mol-%, more preferably at least 95 mol-%, even more preferably at least 98 mol-%, of the repeating units in copolymer (D) are selected from methacrylic acid repeating units, methyl methacrylate repeating units, and methyl acrylate repeating units.

It is preferred that copolymer (D), if present in the first coating, comprises 7 to 13 mol-% methacrylic acid repeating units, 25 to 31 mol-% methyl methacrylate repeating units, and 62 to 68 mol-% methyl acrylate repeating units. The copolymer (D) in the first coating preferably comprises methacrylic acid repeating units, methyl methacrylate repeating units, and methyl acrylate repeating units in a molar ratio of 1:3:7. It is furthermore preferred that the copolymer (D), if present in the first coating, consists of methacrylic acid repeating units, methyl methacrylate repeating units, and methyl acrylate repeating units.

A corresponding preferred example of copolymer (D) is a copolymer comprising (or, more preferably, consisting of) methacrylic acid repeating units, methyl methacrylate repeating units, and methyl acrylate repeating units in a molar ratio of 1:3:7, such as, e.g., Eudragit FS30D.

### Contents of the Respective Copolymers in the First Coating

The content of the copolymer (A) in the first coating is preferably at least 25% (w/w), more preferably at least 50% (w/w), even more preferably at least 75% (w/w), yet even more preferably at least 80% (w/w), still more preferably at least 90% (w/w), in relation to the total weight of the first coating.

As explained above, the first coating comprises the copolymer (A) in combination with the copolymer (B) and/or the copolymer (C) and/or the copolymer (D). It is preferred that the first coating comprises the copolymer (A) in combination with the copolymer (B) and/or the copolymer (D). It is more preferred that the first coating comprises the copolymer (A) and the copolymer (B). In this case, the content of the copolymer (A) in the first coating is preferably at least 25% (w/w), more preferably at least 50% (w/w), even more preferably at least 75% (w/w), yet even more preferably at least 80% (w/w), still more preferably at least 90% (w/w), in relation to the total weight of the copolymer (A) and the copolymer (B) in the first coating.

Alternatively, the first coating may also comprise the copolymer (A) and the copolymer (C). As explained above, the copolymer (C) is preferably a copolymer (C-1) or a copolymer (C-2). Accordingly, the first coating may comprise copolymer (A) and copolymer (C-1), or the first coating may comprise copolymer (A) and copolymer (C-2), or the first coating may comprise copolymer (A), copolymer (C-1) and copolymer (C-2).

When the first coating comprises the copolymer (A) in combination with the copolymer (B), the weight ratio of copolymer (A) to copolymer (B) is preferably within the range of from 9:1 to 1:9 (e.g., 9:1, 8:2, 7:3, 6:4, 5:5, 4:6, 3:7, 2:8, or 1:9). The weight ratio of copolymer (A) to copolymer (B) is more preferably within the range of from 9:1 to 7:3 (e.g., 9:1, 8:2, or 7:3), even more preferably 8:2.

Similarly, when the first coating comprises the copolymer (A) in combination with the copolymer (D), the weight ratio of copolymer (A) to copolymer (D) is preferably within the range of from 9:1 to 1:9 (e.g., 9:1, 8:2, 7:3, 6:4, 5:5, 4:6, 3:7, 2:8, or 1:9). The weight ratio of copolymer (A) to copolymer (D) is more preferably within the range of from 9:1 to 7:3 (e.g., 9:1, 8:2, or 7:3), even more preferably 8:2.

It is to be understood that the first coating may further comprise one or more other polymers, particularly one or more polymers selected from ethyl cellulose, hydroxypropyl methylcellulose (HPMC), and polyvinyl acetate.

The first coating may, for example, account for at least 2% w/w, preferably 2 to 25% w/w, more preferably 3 to 20% w/w, even more preferably 3 to 15% w/w, in relation to the total weight of the solid oral pharmaceutical composition.

The first coating may further comprise one or more plasticizers. The one or more plasticizers are preferably selected from mono-, di- and tri-alkyl citrates such as, e.g., triethyl citrate, tripropyl citrate, tributyl citrate, or acetyl triethyl citrate; dialkyl sebacinates such as, e.g., diethyl sebacinate, dipropyl sebacinate, or dibutyl sebacinate; dialkyl phthalates such as, e.g., dimethyl phthalate, diethyl phthalate, dipropyl phthalate, dibutyl phthalate, or dioctyl phthalate; glycerol and mono-, di- and tri-glycerides such as, e.g., glyceryl triacetate, glyceryl tributyrate, glyceryl monostearate, or acetylated monoglycerides; propylene glycols and polyethylene glycols such as, e.g., PEG 300, PEG 400, PEG 600, PEG 800, PEG 1450, or PEG3350; fatty acids such as, e.g., stearic acid, oleic acid, or esters of fatty acids. More preferably, the one or more plasticizers are selected from mono-, di- and tri-alkyl citrates such as, e.g., triethyl citrate, tripropyl citrate, tributyl citrate or acetyl triethyl citrate. Even more preferably, the first coating further comprises 10 to 80% by weight, preferably 40 to 80% by weight, of one or more selected from triethyl citrate, tripropyl citrate and tributyl citrate, based on the total weight of the first coating. An example of a preferred plasticizer is PlasACRYL, such as PlasACRYL™ HTP20 and PlasACRYL™ T20.

It is preferred that the first coating is obtained from an aqueous dispersion of copolymer (A) and the copolymer (B) and/or copolymer (C) and/or the copolymer (D), optionally further containing any of the optional components of the first coating.

It will be understood that the first coating is exterior to the core which is comprised in the solid oral pharmaceutical composition. The first coating preferably surrounds (or completely covers) and contains the core. While the pharmaceutical composition may also contain one or more intermediate coatings in between the core and the first coating, as described herein below, it is preferred that there is no such intermediate layer, i.e. the first coating preferably is directly exterior to the core (or is in direct contact to the core).

### Optional Second Coating

The solid oral pharmaceutical composition according to the present invention may comprise further coatings (in addition to the above-described first coating). In particular, the solid oral pharmaceutical composition preferably comprises a second coating which is exterior to the first coating, wherein the second coating comprises a copolymer (C). The second coating preferably surrounds (or completely covers) and contains the first coating. Furthermore, the second coating preferably dissolves at a pH in the range of 5 to 7, preferably at a pH in the range of 5.5 to 6.5, more preferably at a pH in the range of 5.5 to 6.0.

The copolymer (C) in the second coating comprises 25 to 60 mol-% methacrylic acid repeating units and 40 to 75 mol-% methyl methacrylate repeating units. The copolymer (C) preferably is an anionic copolymer. Preferably at least 90 mol-%, more preferably at least 95 mol-%, even more preferably at least 98 mol-%, of the repeating units in copolymer (C) are selected from methacrylic acid repeating units and methyl methacrylate repeating units. In particular, the copolymer (C) in the second coating may consist of methacrylic acid repeating units and methyl methacrylate repeating units. The copolymer (C) in the second coating preferably is a copolymer (C-1) or a copolymer (C-2), as described below. Accordingly, the second coating may comprise a copolymer (C-1), a copolymer (C-2), or the combination of both a copolymer (C-1) and a copolymer (C-2).

The copolymer (C-1) in the second coating comprises 25 to 60 mol-% methacrylic acid repeating units and 40 to 75 mol-% methyl methacrylate repeating units, preferably 45 to 55 mol-% methacrylic acid repeating units and 45 to 55 mol-% methyl methacrylate repeating units. Preferably at least 90 mol-%, more preferably at least 95 mol-%, even more preferably at least 98 mol-%, of the repeating units in copolymer (C-1) are selected from methacrylic acid repeating units and methyl methacrylate repeating units. For example, the copolymer (C-1) in the second coating may comprise methacrylic acid repeating units and methyl methacrylate repeating units in a molar ratio of 0.5:1 to 1.5:1, preferably in a molar ratio of 0.8:1 to 1:08, more preferably in a molar ratio of 1:1. In particular, the copolymer (C-1) in the second coating may consist of methacrylic acid repeating units and methyl methacrylate repeating units. A corresponding preferred example of copolymer (C-1) is poly(methacrylic acid-co-methyl methacrylate) 1:1, particularly Eudragit L 100 or Eudragit L 12.5.

The copolymer (C-2) in the second coating comprises 25 to 60 mol-% methacrylic acid repeating units and 40 to 75 mol-% methyl methacrylate repeating units, preferably 25 to 40 mol-% methacrylic acid repeating units and 60 to 75 mol-% methyl methacrylate repeating units. Accordingly, it is preferred that the copolymer (C-2) in the second coating comprises methacrylic acid repeating units and methyl methacrylate repeating units in a molar ratio of 1:1.5 to 1:2.5, more preferably in a molar ratio of 1:2. Preferably at least 90 mol-%, more preferably at least 95 mol-%, even more preferably at least 98 mol-%, of the repeating units in copolymer (C-2) are selected from methacrylic acid repeating units and methyl methacrylate repeating units. In particular, the copolymer (C-2) in the second coating may consist of methacrylic acid repeating units and methyl methacrylate repeating units. A corresponding preferred example of copolymer (C-2) is poly(methacrylic acid-co-methyl methacrylate) 1:2, particularly Eudragit S 100.

It is generally preferred that the copolymer (C) in the second coating, including the copolymer (C-1) and/or the copolymer (C-2), does not comprise methyl acrylate repeating units. Thus, the copolymer (C) in the second coating (or the copolymer (C-1) and/or the copolymer (C-2)) preferably comprises not more than 3 mol-% methyl acrylate repeating units, more preferably not more than 1 mol-%, even more preferably not more than 0.5 mol-%, yet even more preferably not more than 0.1 mol-%, still more preferably not more than 0.01 mol-%, most preferably 0 mol-% methyl acrylate repeating units.

It is preferred that the copolymer (C) in the second coating, including the copolymer (C-1) and/or the copolymer (C-2), is obtained from an aqueous dispersion of the respective copolymer (i.e., copolymer (C), copolymer (C-1) or copolymer (C-2)).

The second coating may further comprise one or more plasticizers. The one or more plasticizers are preferably selected from mono-, di- and tri-alkyl citrates such as, e.g., triethyl citrate, tripropyl citrate, tributyl citrate, or acetyl triethyl citrate; dialkyl sebacinates such as, e.g., diethyl sebacinate, dipropyl sebacinate, or dibutyl sebacinate; dialkyl phthalates such as, e.g., dimethyl phthalate, diethyl phthalate, dipropyl phthalate, dibutyl phthalate, or dioctyl phthalate; glycerol and mono-, di- and tri-glycerides such as, e.g., glyceryl triacetate, glyceryl tributyrate, glyceryl monostearate, or acetylated monoglycerides; propylene glycols and polyethylene glycols such as, e.g., PEG 300, PEG 400, PEG 600, PEG 800, PEG 1450, or PEG3350; fatty acids such as, e.g., stearic acid, oleic acid, or esters of fatty acids. More preferably, the one or more plasticizers are selected from mono-, di- and tri-alkyl citrates such as, e.g., triethyl citrate, tripropyl citrate, tributyl citrate, or acetyl triethyl citrate. Even more preferably, the second coating further comprises 10 to 80% by weight, preferably 40 to 80% by weight, of one or more selected from triethyl citrate, tripropyl citrate and tributyl citrate, based on the total weight of the second coating. An example of a preferred plasticizer is PlasACRYL, such as PlasACRYL™ HTP20 and PlasACRYL™ T20.

The second coating may, for example, account for at least 0.1% w/w, preferably 0.5 to 8% w/w, more preferably 1 to 5% w/w, in relation to the total weight of the solid oral pharmaceutical composition.

### Optional Intermediate Coating

The solid oral pharmaceutical composition may further comprise one or more intermediate coatings located between the core and the first coating. The intermediate coating, or the innermost intermediate coating (in the case of more than one intermediate coating), may be a substantially continuous layer surrounding and containing the core of the solid oral pharmaceutical composition. Each intermediate coating (if present) preferably constitutes 5% w/w or less, more preferably 2% w/w or less, even more preferably 1% w/w or less of the solid oral pharmaceutical composition. Moreover, each intermediate coating (if present) preferably constitutes 0.1% w/w or more, more preferably 0.5% w/w or more of the solid oral pharmaceutical composition. The invention relates to all combinations of the aforementioned minimum and maximum contents by weight of the intermediate coating(s).

Each intermediate coating preferably comprises one or more polymers selected from ethyl cellulose, hydroxypropyl methylcellulose (HPMC), and polyvinyl acetate. More preferably, there is only one intermediate coating, and said intermediate coating comprises hydroxypropyl methylcellulose (HPMC). Even more preferably, there is only one intermediate coating, and said intermediate coating consists of hydroxypropyl methylcellulose (HPMC).

### Optional Third Coating

The solid oral pharmaceutical composition may further comprise a third coating surrounding and containing the second coating (if present) or the first coating (if no second coating is present).

The third coating, if present, preferably accounts for at least 0.1% w/w, more preferably 0.5 to 8% w/w, even more preferably 1 to 5% w/w, of the total weight of the solid oral pharmaceutical composition.

The composition of the third coating is not particularly limited. Preferably, the third coating contains one or more copolymers selected from copolymers (A), (B), (C) and (D) as defined herein. More preferably, it comprises a cationic copolymer based on dimethylaminoethyl methacrylate, butyl methacrylate, and methyl methacrylate, preferably in a molar ratio of 2:1:1 (such as Eudragit E 100 or other Eudragit E polymers).

Alternatively, the third coating may be a top coat. The top coat may be a film coating or an immediate release coating. Examples of suitable top coats include Opadry® White (obtainable from Colorcon, Pa., USA), Opadry® II Yellow (obtainable from Colorcon, Pa., USA), or a copolymer based on methacrylic acid and ethyl acrylate, such as, e.g., a copolymer comprising at least 40% methacrylic acid repeating units and at least 40% ethyl acrylate repeating units. For example, the third coating may comprise poly(methacrylic acid-co-ethyl acrylate) 1:1.

### The Peptide or Protein Drug

The solid oral pharmaceutical composition of the present invention comprises a core which contains a peptide or protein drug. It is preferred that the peptide or protein drug is present only in the core, i.e. that it is not present in any coating comprised in the solid oral pharmaceutical composition.

The peptide or protein drug preferably has a molecular weight of equal to or less than about 300 kDa (such as, e.g., equal to or less than about 260 kDa, or equal to or less than about 220 kDa, or equal to or less than about 180 kDa, or equal to or less than about 150 kDa, or equal to or less than about 120 kDa, or equal to or less than about 100 kDa, or equal to or less than about 90 kDa, or equal to or less than about 80 kDa, or equal to or less than about 70 kDa, or equal to or less than about 60 kDa, or equal to or less than about 50 kDa, or equal to or less than about 40 kDa, or equal to or less than about 30 kDa, or equal to or less than about 20 kDa, or equal to or less than about 10 kDa, or equal to or less than about 5 kDa, or equal to or less than about 2 kDa, or equal to or less than about 1 kDa, or equal to or less than about 500 Da). More preferably, the peptide or protein drug has a maximum molecular weight of equal to or less than about 200 kDa, even more preferably equal to or less than about 150 kDa, even more preferably equal to or less than about 100 kDa, even more preferably equal to or less than about 50 kDa, even more preferably equal to or less than about 40 kDa, even more preferably equal to or less than about 30 kDa, even more preferably equal to or less than about 20 kDa, and yet even more preferably equal to or less than about 10 kDa. It is furthermore preferred that the peptide or protein drug has a minimum molecular weight of equal to or greater than about 300 Da, more preferably equal to or greater than about 500 Da, even more preferably equal to or greater than about 800 Da, and yet even more preferably equal to or greater than about 1 kDa. Accordingly, it is particularly preferred that the peptide or protein drug has a molecular weight of about 300 Da to about 150 kDa, more preferably about 300 Da to about 50 kDa, even more preferably about 500 Da to about 30 kDa, even more preferably about 500 Da to about 20 kDa, even more preferably about 800 Da to about 10 kDa, and yet even more preferably about 1 kDa to about 6 kDa.

The molecular weight of the peptide or protein drug is indicated herein in dalton (Da), which is an alternative name for the unified atomic mass unit (u). A molecular weight of, e.g., 500 Da is thus equivalent to 500 g/mol. The term “kDa” (kilodalton) refers to 1000 Da.

The molecular weight of the peptide or protein drug can be determined using methods known in the art, such as, e.g., mass spectrometry (e.g., electrospray ionization mass spectrometry (ESI-MS) or matrix-assisted laser desorption/ionization mass spectrometry (MALDI-MS)), gel electrophoresis (e.g., polyacrylamide gel electrophoresis using sodium dodecyl sulfate (SDS-PAGE)), hydrodynamic methods (e.g., gel filtration chromatography or gradient sedimentation), or static light scattering (e.g., multi-angle light scattering (MALS)). It is preferred that the molecular weight of the peptide or protein drug is determined using mass spectrometry.

The peptide or protein drug may be any peptide or protein that is suitable to be used as a medicament. For example, the peptide or protein drug may be a linear peptide or protein drug or a cyclic peptide or protein drug (e.g., a cyclic peptide or protein drug that is cyclized via at least one ester linkage and/or at least one amide linkage; such as, e.g., a cyclotide; cyclotides are disulfide rich peptides characterized by their head-to-tail cyclized peptide backbone and the interlocking arrangement of their disulfide bonds). It may also be a modified or derivatized peptide or protein drug, such as a PEGylated peptide or protein drug or a fatty acid acylated peptide or protein drug or a fatty diacid acylated peptide or protein drug. Moreover, the peptide or protein drug may be free of histidine residues and/or free of cysteine residues. It is generally preferred that the peptide or protein drug is water-soluble, particularly at neutral pH (i.e., at about pH 7). The invention also allows the use of a peptide or protein drug that has at least one serine protease cleavage site, i.e., a peptide or protein drug which comprises one or more amino acid residue(s) amenable or prone to cleavage by a serine protease. The term “peptide or protein drug” is used herein synonymously with “therapeutic peptide or protein” and “therapeutic peptide or protein drug”.

The peptide or protein drug is preferably selected from insulin (preferably human insulin), an insulin analog (e.g., a long acting basal insulin analog or a protease stabilized long acting basal insulin analog; exemplary insulin analogs include, without limitation, insulin lispro, insulin PEGlispro, the insulin derivative “A14E, B25H, B29K(N(eps)octadecanedioyl-gGlu-OEG-OEG), desB30 human insulin” (see, e.g., US 2014/0056953 A1), insulin aspart, insulin glulisine, insulin glargine, insulin detemir, NPH insulin, insulin degludec, and the insulin analogs/derivatives described in US 2014/0056953 A1, which is incorporated herein by reference, particularly each one of the insulin analogs/derivatives described in paragraphs [0225] to [0332] of US 2014/0056953 A1), GLP-2, a GLP-2 agonist or analog (e.g., apraglutide, teduglutide or elsiglutide), glucose-dependent insulinotropic polypeptide (also referred to as “gastric inhibitory polypeptide” or GIP), a gastric inhibitory polypeptide (GIP) receptor agonist, elamipretide, a cyclotide (i.e., a peptide characterized by its head-to-tail cyclized peptide backbone and the interlocking arrangement of its disulfide bonds; including, e.g., a cyclotide having at least two disulfide bonds, and preferably a cyclotide having three disulfide bonds), recombinant factor VIIa (rFVIIa), eptacog alfa, amylin, an amylin analog, pramlintide, a somatostatin analog (e.g., octreotide, lanreotide, or pasireotide), goserelin (e.g., goserelin acetate), buserelin (e.g., buserelin acetate), leptin, a leptin analog (e.g., metreleptin), peptide YY (PYY), a PYY analog, glatiramer (e.g., glatiramer acetate), leuprolide (e.g., leuprolide acetate), desmopressin (e.g., desmopressin acetate, particularly desmopressin monoacetate trihydrate), a desmopressin analog, a vasopressin receptor 2 (V2 receptor) agonist peptide, osteocalcin, an osteocalcin analog or derivative, human growth hormone (hGH), a human growth hormone analog, a long-acting human growth hormone (such as, e.g., somapacitan or hGH-CTP (human growth hormone derivatized with the C-terminal peptide (CTP) of the beta chain of human chorionic gonadotropin (hCG)), fibroblast growth factor 21 (FGF21), an antibody (e.g., any of the exemplary antibodies described herein below), a glycopeptide antibiotic (e.g., a glycosylated cyclic or polycyclic nonribosomal peptide, such as vancomycin, teicoplanin, telavancin, bleomycin, ramoplanin, or decaplanin), a cyclotide, bortezomib, cosyntropin, chorionic gonadotropin, menotropin, sermorelin, luteinizing-hormone-releasing hormone (LHRH; also referred to as “gonadotropin-releasing hormone”), somatropin, calcitonin (e.g., calcitonin-salmon), pentagastrin, oxytocin, nesiritide, anakinra, enfuvirtide, pegvisomant, dornase alfa, lepirudin, anidulafungin, eptifibatide, interferon alfacon-1, interferon alpha-2a, interferon alpha-2b, interferon beta-1a, interferon beta-1b, interferon gamma-1b, peginterferon alfa-2a (i.e., pegylated interferon alfa-2a), peginterferon alfa-2b (i.e., pegylated interferon alfa-2b), peginterferon beta-1a (i.e., pegylated interferon beta-1a), fibrinolysin, vasopressin, aldesleukin, an epoetin, epoetin alfa, darbepoetin alfa, epoetin beta, epoetin delta, epoetin omega, epoetin zeta, epoetin theta, methoxy polyethylene glycol-epoetin beta, continuous erythropoietin receptor activator (CERA; a pegylated EPO derivative), pegylated epo, albupoetin, an epo-dimer analogue, epo-Fc, carbamylated EPO (CEPO), synthetic erythropoese protein (SEP), low molecular epo analogue (PBI-1402), filgrastim, PEG-filgrastim, interleukin-11, interleukin-23 receptor antagonist peptide, cyclosporine, glucagon, urokinase, viomycin, thyrotropin-releasing hormone (TRH), leucine-enkephalin, methionine-enkephalin, substance P (CAS no. 33507-63-0), adrenocorticotropic hormone (ACTH), parathyroid hormone (PTH), a parathyroid hormone (PTH) fragment (e.g., teriparatide (also referred to as “PTH(1-34)”), PTH(1-31), PTH(2-34)) or eneboparatide, parathyroid hormone-related protein (PTHrP), abaloparatide, linaclotide, carfilzomib, icatibant, ecallantide, cilengitide, a prostaglandin F2a receptor modulator (e.g., PDC31), abciximab (C7E3-Fab), ranibizumab, alefacept, romiplostim, anakinra, abatacept, belatacept, and pharmaceutically acceptable salts thereof. If the subject/patient to be treated is a human and if the peptide or protein drug is an endogenous peptide or protein in human beings (i.e., occurs naturally in humans; such as, e.g., insulin or glucagon), it is furthermore preferred to use a human isoform of the corresponding peptide or protein (which may, e.g., be recombinantly expressed or chemically synthesized).

More preferably, the peptide or protein drug is selected from GLP-2, a GLP-2 agonist or analog (e.g., apraglutide, teduglutide or elsiglutide), insulin (particularly human insulin), an insulin analog (e.g., a long acting basal insulin analog or a protease stabilized long acting basal insulin analog; exemplary insulin analogs include, without limitation, insulin lispro, insulin PEGlispro, the insulin derivative “A14E, B25H, B29K(N(eps)octadecanedioyl-gGlu-OEG-OEG), desB30 human insulin” (see, e.g., US 2014/0056953 A1), insulin aspart, insulin glulisine, insulin glargine, insulin detemir, NPH insulin, insulin degludec, and the insulin analogs/derivatives described in US 2014/0056953 A1, which is incorporated herein by reference, particularly each one of the insulin analogs/derivatives described in paragraphs [0225] to [0332] of US 2014/0056953 A1), an antibody, recombinant factor VIIa (rFVIIa), eptacog alfa, amylin, an amylin analog, pramlintide, a somatostatin analog (e.g., octreotide, lanreotide, or pasireotide), goserelin (e.g., goserelin acetate), buserelin, peptide YY (PYY), a PYY analog, glatiramer (e.g., glatiramer acetate), leuprolide (e.g., leuprolide acetate), desmopressin (e.g., desmopressin acetate, particularly desmopressin monoacetate trihydrate), a desmopressin analog, a vasopressin receptor 2 (V2 receptor) agonist peptide, teicoplanin, telavancin, bleomycin, ramoplanin, decaplanin, bortezomib, cosyntropin, sermorelin, luteinizing-hormone-releasing hormone (LHRH), calcitonin (e.g., calcitonin-salmon), pentagastrin, nesiritide, enfuvirtide, eptifibatide, cyclosporine, glucagon, viomycin, thyrotropin-releasing hormone (TRH), leucine-enkephalin, methionine-enkephalin, substance P, a parathyroid hormone (PTH) fragment (e.g., teriparatide (PTH(1-34)), PTH(1-31), PTH(2-34)) or eneboparatide, carfilzomib, icatibant, cilengitide, a prostaglandin F2a receptor modulator (e.g., PDC31), and pharmaceutically acceptable salts thereof.

Even more preferably, the peptide or protein drug is selected from GLP-2, a GLP-2 agonist or analog (e.g., apraglutide, teduglutide or elsiglutide), insulin (e.g., human insulin), an insulin analog (e.g., insulin lispro, insulin PEGlispro, “A14E, B25H, B29K(N(eps)octadecanedioyl-gGlu-OEG-OEG), desB30 human insulin”, insulin aspart, insulin glulisine, insulin glargine, insulin detemir, NPH insulin, or insulin degludec), an antibody, a somatostatin analog (e.g., octreotide, lanreotide, or pasireotide), leuprolide (e.g., leuprolide acetate), desmopressin (e.g., desmopressin acetate, particularly desmopressin monoacetate trihydrate), a desmopressin analog, a vasopressin receptor 2 (V2 receptor) agonist peptide, a parathyroid hormone (PTH) fragment (e.g., teriparatide (PTH(1-34)), PTH(1-31), PTH(2-34)) or eneboparatide, and pharmaceutically acceptable salts thereof.

Yet even more preferably, the peptide or protein drug is selected from GLP-2, a GLP-2 agonist or analog (e.g., apraglutide, teduglutide or elsiglutide), insulin (e.g., human insulin), an insulin analog (e.g., insulin lispro, insulin PEGlispro, “A14E, B25H, B29K(N(eps)octadecanedioyl-gGlu-OEG-OEG), desB30 human insulin”, insulin aspart, insulin glulisine, insulin glargine, insulin detemir, NPH insulin, or insulin degludec), an antibody, a somatostatin analog (e.g., octreotide, lanreotide, or pasireotide), desmopressin (e.g., desmopressin acetate, particularly desmopressin monoacetate trihydrate), a desmopressin analog, a vasopressin receptor 2 (V2 receptor) agonist peptide, a parathyroid hormone (PTH) fragment (e.g., teriparatide (PTH(1-34)), PTH(1-31), PTH(2-34)) or eneboparatide, and pharmaceutically acceptable salts thereof.

Still more preferably, the peptide or protein drug is selected from GLP-2, a GLP-2 agonist or analog (e.g., apraglutide, teduglutide or elsiglutide), insulin (e.g., human insulin), an insulin analog (e.g., insulin lispro, insulin PEGlispro, “A14E, B25H, B29K(N(eps)octadecanedioyl-gGlu-OEG-OEG), desB30 human insulin”, insulin aspart, insulin glulisine, insulin glargine, insulin detemir, NPH insulin, or insulin degludec), and an antibody (particularly a therapeutic antibody for the treatment or prevention of a local intestinal disease/disorder, such as an intestinal inflammatory disorder; e.g., ustekinumab or adalimumab).

As noted above, the peptide or protein drug may also be an insulin analog. Preferred examples of an insulin analog include, in particular, the following:
B29K(N(ε)hexadecanedioyl-γ-L-Glu) A14E B25H desB30 human insulin;
B29K(N(ε)octadecanedioyl-γ-L-Glu-OEG-OEG) desB30 human insulin;
B29K(N(ε)octadecanedioyl-γ-L-Glu) A14E B25H desB30 human insulin;
B29K(N(ε)eicosanedioyl-γ-L-Glu) A14E B25H desB30 human insulin;
B29K(N(ε)octadecanedioyl-γ-L-Glu-OEG-OEG) A14E B25H desB30 human insulin;
B29K(N(ε)eicosanedioyl-γ-L-Glu-OEG-OEG) A14E B25H desB30 human insulin;
B29K(N(ε)eicosanedioyl-γ-L-Glu-OEG-OEG) A14E B16H B25H desB30 human insulin;
B29K(N(ε)hexadecanedioyl-γ-L-Glu) A14E B16H B25H desB30 human insulin;
B29K(N(ε)eicosanedioyl-γ-L-Glu-OEG-OEG) A14E B16H B25H desB30 human insulin; or
B29K(N(ε)octadecanedioyl) A14E B25H desB30 human insulin.

These insulin analogs are described and characterized, e.g., in US 2014/0056953 A1. It is particularly preferred that the insulin analog is B29K(N(ε)octadecanedioyl-γ-L-Glu-OEG-OEG) A14E B25H desB30 human insulin.

As described above, the peptide or protein drug may also be an antibody, preferably a monoclonal antibody, and it may in particular be a single-chain antibody or a single-domain antibody (e.g., a “nanobody”). An example of a nanobody, which can be used as the peptide or protein drug in accordance with the invention, is caplacizumab. Caplacizumab is a single-domain antibody which can be used, e.g., in the treatment or prevention of thrombotic thrombocytopenic purpura (TPP) or of thrombosis.

In particular, the peptide or protein drug may be an antibody selected from 3F8, 8H9, abagovomab, abciximab, abituzumab, abrezekimab, abrilumab, actoxumab, adalimumab, adecatumumab, atidortoxumab, aducanumab, afasevikumab, afelimomab, afutuzumab, alacizumab pegol, alemtuzumab, alirocumab, altumomab pentetate, amatuximab, anatumomab mafenatox, andecaliximab, anetumab ravtansine, anifrolumab, anrukinzumab, apolizumab, aprutumab ixadotin, arcitumomab, ascrinvacumab, aselizumab, atezolizumab, atinumab, atorolimumab, avelumab, azintuxizumab vedotin, bapineuzumab, basiliximab, bavituximab, BCD-100, bectumomab, begelomab, belantamab mafodotin, belimumab, bemarituzumab, benralizumab, berlimatoxumab, bersanlimab, bertilimumab, besilesomab, bevacizumab, bezlotoxumab, biciromab, bimagrumab, bimekizumab, birtamimab, bivatuzumab mertansine, BIVV009, bleselumab, blinatumomab, blontuvetmab, blosozumab, bococizumab, brazikumab, brentuximab vedotin, briakinumab, brodalumab, brolucizumab, brontictuzumab, burosumab, cabiralizumab, camidanlumab tesirine, camrelizumab, canakinumab, cantuzumab mertansine, cantuzumab ravtansine, caplacizumab, capromab pendetide, carlumab, carotuximab, catumaxomab, cbr96-doxorubicin immunoconjugate, cedelizumab, cemiplimab, cergutuzumab amunaleukin, certolizumab pegol, cetrelimab, cetuximab, cibisatamab, citatuzumab bogatox, cixutumumab, clazakizumab, clenoliximab, clivatuzumab tetraxetan, codrituzumab, cofetuzumab pelidotin, coltuximab ravtansine, conatumumab, concizumab, cosfroviximab, crenezumab, crizanlizumab, crotedumab, CR6261, cusatuzumab, dacetuzumab, daclizumab, dalotuzumab, dapirolizumab pegol, daratumumab, dectrekumab, demcizumab, denintuzumab mafodotin, denosumab, depatuxizumab mafodotin, derlotuximab biotin, detumomab, demcizumab, dinutuximab, diridavumab, domagrozumab, dorlimomab aritox, drozitumab, DS-8201, duligotuzumab, dupilumab, durvalumab, dusigitumab, duvortuxizumab, ecromeximab, eculizumab, edobacomab, edrecolomab, efalizumab, efungumab, eldelumab, elezanumab, elgemtumab, elotuzumab, elsilimomab, emactuzumab, emapalumab, emibetuzumab, emicizumab, enapotamab edotin, enavatuzumab, enfortumab vedotin, enlimomab pegol, enoblituzumab, enokizumab, enoticumab, ensituximab, epitumomab cituxetan, epratuzumab, eptinezumab, erenumab, erlizumab, ertumaxomab, etaracizumab, etigilimab, etrolizumab, evinacumab, evolocumab, exbivirumab, fanolesomab, faralimomab, faricimab, farletuzumab, fasinumab, FBTA05, felvizumab, fezakinumab, fibatuzumab, ficlatuzumab, figitumumab, firivumab, flanvotumab, fletikumab, flotetuzumab, fontolizumab, foralumab, foravirumab, fremanezumab, fresolimumab, frunevetmab, fulranumab, futuximab, galcanezumab, galiximab, gancotamab, ganitumab, gantenerumab, gatipotuzumab, gavilimomab, gedivumab, gemtuzumab ozogamicin, gevokizumab, gilvetmab, gimsilumab, girentuximab, glembatumumab vedotin, golimumab, gomiliximab, gosuranemab, guselkumab, ianalumab, ibalizumab, IB1308, ibritumomab tiuxetan, icrucumab, idarucizumab, ifabotuzumab, igovomab, iladatuzumab vedotin, IMAB362, imalumab, imaprelimab, imciromab, imgatuzumab, inclacumab, indatuximab ravtansine, indusatumab vedotin, inebilizumab, infliximab, intetumumab, inolimomab, inotuzumab ozogamicin, ipilimumab, iomab-b, iratumumab, isatuximab, iscalimab, istiratumab, itolizumab, ixekizumab, keliximab, labetuzumab, lacnotuzumab, ladiratuzumab vedotin, lampalizumab, lanadelumab, landogrozumab, laprituximab emtansine, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab vedotin, ligelizumab, loncastuximab tesirine, losatuxizumab vedotin, lilotomab satetraxetan, lintuzumab, lirilumab, lodelcizumab, lokivetmab, lorvotuzumab mertansine, lucatumumab, lulizumab pegol, lumiliximab, lumretuzumab, lupartumab amadotin, lutikizumab, MABp1, mapatumumab, margetuximab, marstacimab, maslimomab, mavrilimumab, matuzumab, mepolizumab, metelimumab, milatuzumab, minretumomab, mirikizumab, mirvetuximab soravtansine, mitumomab, modotuximab, mogamulizumab, monalizumab, morolimumab, mosunetuzumab, motavizumab, moxetumomab pasudotox, muromonab-CD3, nacolomab tafenatox, namilumab, naptumomab estafenatox, naratuximab emtansine, narnatumab, natalizumab, navicixizumab, navivumab, naxitamab, nebacumab, necitumumab, nemolizumab, NEOD001, nerelimomab, nesvacumab, netakimab, nimotuzumab, nirsevimab, nivolumab, nofetumomab merpentan, obiltoxaximab, obinutuzumab, ocaratuzumab, ocrelizumab, odulimomab, ofatumumab, olaratumab, oleclumab, olendalizumab, olokizumab, omalizumab, OMS721, onartuzumab, ontuxizumab, onvatilimab, opicinumab, oportuzumab monatox, oregovomab, orticumab, otelixizumab, otilimab, otlertuzumab, oxelumab, ozanezumab, ozoralizumab, pagibaximab, palivizumab, pamrevlumab, panitumumab, pankomab, panobacumab, parsatuzumab, pascolizumab, pasotuxizumab, pateclizumab, patritumab, PDR001, pembrolizumab, pemtumomab, perakizumab, pertuzumab, pexelizumab, pidilizumab, pinatuzumab vedotin, pintumomab, placulumab, plozalizumab, pogalizumab, polatuzumab vedotin, ponezumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, anibizumab, raxibacumab, ravagalimab, ravulizumab, refanezumab, regavirumab, remtolumab, reslizumab, rilotumumab, rinucumab, risankizumab, rituximab, rivabazumab pegol, robatumumab, rmab, roledumab, romilkimab, romosozumab, rontalizumab, rosmantuzumab, rovalpituzumab tesirine, rovelizumab, rozanolixizumab, ruplizumab, SA237, sacituzumab govitecan, samalizumab, samrotamab vedotin, sapelizumab, sarilumab, satralizumab, satumomab pendetide, secukinumab, selicrelumab, seribantumab, setoxaximab, setrusumab, sevirumab, sibrotuzumab, SGN-CD19A, SHP647, sifalimumab, siltuximab, simtuzumab, siplizumab, sirtratumab vedotin, sirukumab, sofituzumab vedotin, solanezumab, solitomab, sonepcizumab, sontuzumab, spartalizumab, stamulumab, sulesomab, suptavumab, sutimlimab, suvizumab, suvratoxumab, tabalumab, tacatuzumab etraxetan, tadocizumab, talacotuzumab, talizumab, tamtuvetmab, tanezumab, taplitumomab paptox, tarextumab, tavolimab, tefibazumab, telimomab aritox, telisotuzumab vedotin, tenatumomab, teneliximab, teplizumab, tepoditamab, teprotumumab, tesidolumab, tetulomab, tezepelumab, TGN1412, tibulizumab, tildrakizumab, tigatuzumab, timigutuzumab, timolumab, tiragotumab, tislelizumab, tisotumab vedotin, TNX-650, tocilizumab, tomuzotuximab, toralizumab, tosatoxumab, tositumomab, tovetumab, tralokinumab, trastuzumab, trastuzumab emtansine, TRBS07, tregalizumab, tremelimumab, trevogrumab, tucotuzumab celmoleukin, tuvirumab, ublituximab, ulocuplumab, urelumab, urtoxazumab, ustekinumab, utomilumab, vadastuximab talirine, vanalimab, vandortuzumab vedotin, vantictumab, vanucizumab, vapaliximab, varisacumab, varlilumab, vatelizumab, vedolizumab, veltuzumab, vepalimomab, vesencumab, visilizumab, vobarilizumab, volociximab, vonlerolizumab, vopratelimab, vorsetuzumab mafodotin, votumumab, vunakizumab, xentuzumab, XMAB-5574, zalutumumab, zanolimumab, zatuximab, zenocutuzumab, ziralimumab, zolbetuximab, and zolimomab aritox.

Moreover, it is particularly preferred that the peptide or protein drug is an anti-obesity peptide (which may also be referred to as a “peptidic anti-obesity agent”). Preferred examples of anti-obesity peptides include any of the following:
a neuropeptide Y receptor (NPY) agonist peptide, such as e.g. a NPY receptor Y1, Y2, Y4 or Y5 agonist peptide or a pancreatic polypeptide receptor agonist peptide, such as e.g. neuropeptide Y, peptide YY (i.e., “PYY” or peptide tyrosine tyrosine), PYY₃₋₃₆, a PYY analog or derivative (e.g., a long acting fatty acid acylated PYY analog), a neuropeptide FF receptor type 2 (NPFF2R) agonist, a G-protein coupled receptor 10 (GPR10) agonist, a fatty acid acylated dual GPR10-NPFF2R co-agonist, pancreatic polypeptide, prolactin releasing peptide (PrRP), a long acting PrRP31 analog, a C18 lipidated PrRP31 analog (which may be used, in particular, for the treatment of obesity or appetite regulation), GT001 (Gila Therapeutics), or PYY-1875 (Novo Nordisk);
a leptin receptor agonist peptide (LEP-R), such as e.g. leptin, a leptin analog or derivative, such as e.g. a long acting fatty acid acylated leptin analog;
a ghrelin receptor antagonist peptide;
amylin (i.e., islet amyloid polypeptide (IAPP)), an amylin analog or derivative, such as e.g. a long acting fatty acid acylated amylin analog, pramlintide, cagrilintide, or ZP8396;
a gastric inhibitory polypeptide (GIP) receptor agonist peptide, such as e.g. a gastric inhibitory polypeptide (GIP) analog or derivative, such as e.g. a long-acting acylated GIP analog, a GIP agonist, a dual- or tri-agonist GIP peptide, or ZP6590; or
a glucagon receptor agonist peptide, such as e.g. glucagon or a glucagon analog or derivative, such as e.g. a long-acting acylated glucagon analog.

The peptide or protein drug may further be a GLP-2 receptor agonist peptide, such as e.g. GLP-2, a GLP-2 analog or derivative, such as e.g. a long-acting acylated GLP-2 analog, teduglutide, glepaglutide, apraglutide, dapiglutide, or elsiglutide. Such peptide or protein drugs can be used, e.g., in the treatment or prevention of short bowel syndrome (SBS).

The peptide or protein drug may further be, e.g., forigerimod, EA-230, difelikefalin acetate, an agonist of κ-opioid receptor (KOR), aviptadil, a zonulin antagonist, larazotide, brimapitide, a Small Integrin-Binding Ligand N-linked Glycoprotein (SIBLING), TPX-100, ZP9830, a Kv1.3 ion channel blocker, ZP10000, an α4β7 integrin inhibitor, a pellino-1 protein-protein interaction inhibitor peptide, BBT-401 (which can be used, e.g., for the treatment or prevention of ulcerative colitis), an alpha-4-beta-7 (α4β7) integrin antagonist, PN-943, an interleukin (IL) receptor targeted peptide, an IL-23 receptor targeted peptide, PN-235, PN-232, an IL-23 receptor antagonist, or JNJ-77242113. The aforementioned peptide or protein drugs may be used, e.g., in the treatment of prevention of an inflammatory or autoimmune disorder, including e.g. inflammatory bowel disease (IBD). Furthermore, the peptide or protein drug may also be a “nanobody” or an engineered antibody derived from a “heavy-chain-only” peptide or protein, such as e.g. an anti-IL13/OX40L nanobody, an anti-IL-6R nanobody, vobarilizumab, a single-domain antibody, or caplacizumab.

The peptide or protein drug may further be, e.g., dolcanatide, a WNT5A-mimicking peptide, Foxy-5, a peptide that induces thrombospondin-1 (Tsp-1) expression in the tumor microenvironment, cyclic pentapeptide VT1021, an antagonist of the chemokine receptor CXCR4, balixafortide, an anticytokine peptide, BNZ132-1-40 (a 19-mer pegylated peptide), a FK506-binding protein like (FKBPL) peptide, ALM-201 (a 23-mer peptide drug), fexapotide triflutate, the tripeptide tyroserleutide, a luteinizing hormone-releasing hormone (LHRH) antagonist acting on gonadotropin-releasing hormone (GnRH) receptors, ozarelix, an LHRH natural ligand derivative, EP-100, a somatostatin receptor agonist, or HTL0030310. Such peptide or protein drugs can be used, in particular, in the treatment or prevention of cancer.

The peptide or protein drug may further be, e.g., vosoritide (which can be used, in particular, for the treatment or prevention of achondroplasia), a relaxin receptor modulator peptide, relaxin, a relaxin analog or derivative, a long-acting acylated relaxin analog, serelaxin, davunetide (or NAP or AL-108), zilucoplan, or alirinetide (or GM604 or GM6).

Moreover, the peptide or protein drug may also be a GLP-1 receptor agonist, such as, e.g., semaglutide, liraglutide, exenatide, albiglutide, dulaglutide, lixisenatide, taspoglutide, langlenatide, beinaglutide, efpeglenatide, GLP-1(7-37), GLP-1(7-36)NH₂, oxyntomodulin, an oxyntomodulin derivative or analog, a dual GLP-1 receptor/glucagon receptor agonist, a dual GLP-1 receptor/gastric inhibitory peptide (GIP) receptor agonist, or a triple GLP-1 receptor/GIP receptor/glucagon receptor agonist. However, it is preferred that the peptide or protein drug is not a GLP-1 receptor agonist (or is not any of the aforementioned specific GLP-1 receptor agonists). The present invention thus preferably relates to a solid oral pharmaceutical composition, as described and defined herein, in which the peptide or protein drug is not a GLP-1 receptor agonist.

The peptide or protein drug to be used in accordance with the invention can also be a mixture of two or more different peptide or protein drugs, including any of the above-mentioned specific peptide or protein drugs. For example, as the peptide or protein drug, it may be preferable that not only one of semaglutide and octreotide, but a mixture of these be used. As a further example, it may be preferable that not only one of semaglutide, octreotide and leuprolide is used as the peptide or protein drug, but that a mixture of these be used.

The above-described exemplary peptide or protein drugs have been proposed in the literature to be suitable for the treatment or prevention of various different diseases/disorders, and some of these peptide or protein drugs have already received marketing authorizations for specific therapeutic indications. The present invention also specifically relates to the solid oral pharmaceutical composition provided herein for use in the treatment or prevention of a disease/disorder that is amenable to be treated or prevented with the respective peptide or protein drug. Likewise, the invention relates to a method of treating or preventing a disease/disorder, the method comprising orally administering, to a subject in need thereof, the solid oral pharmaceutical composition provided herein, wherein said disease/disorder is a disease/disorder that is amenable to be treated or prevented with the respective peptide or protein drug. Preferred examples of diseases/disorders that are amenable to be treated or prevented with any particular peptide or protein drug in accordance with the present invention are disclosed in (and can be derived from) the medical literature, particularly from any one of: ROTE LISTE online edition (in the version as of the priority date or the filing date of the present specification, particularly as of Sep. 7, 2020); ROTE LISTE 2020 (print edition, Rote Liste Service GmbH, 2020, ISBN 978-3-946057-52-9); GELBE LISTE online edition (in the version as of the priority date or the filing date of the present specification, particularly as of Sep. 7, 2020); GELBE LISTE 2017 (print edition, AVOXA—Mediengruppe Deutscher Apotheker GmbH, 2017, ISBN 978-3774199149); Aktories K et al. (eds.), Allgemeine und spezielle Pharmakologie und Toxikologie, Urban & Fischer Verlag/Elsevier GmbH, 12^{th} edition, 2017, ISBN 978-3437425257; Ammon HPT et al. (eds.), Hunnius Pharmazeutisches Wörterbuch, De Gruyter, 11^{th} edition, 2014, ISBN 978-3110309904; Otto H H et al., Arzneimittel—Ein Handbuch for Arzte und Apotheker, Wissenschaftliche Verlagsgesellschaft, 2017, ISBN 978-3804737266; DrugBank (www.drugbank.ca, in the version as of the priority date or the filing date of the present specification, particularly as of Sep. 7, 2020); Drugs.com (www.drugs.com, in the version as of the priority date or the filing date of the present specification, particularly as of Sep. 7, 2020); Merck Manuals (www.merckmanuals.com, in the version as of the priority date or the filing date of the present specification, particularly as of Sep. 7, 2020); European Medicines Agency (EMA) database (www.ema.europa.eu, in the version as of the priority date or the filing date of the present specification, particularly as of Sep. 7, 2020); U.S. Food & Drug Administration (U.S. FDA) database (www.fda.gov, in the version as of the priority date or the filing date of the present specification, particularly as of Sep. 7, 2020); Japanese Pharmaceuticals and Medical Devices Agency database (www.pmda.go.jp, in the version as of the priority date or the filing date of the present specification, particularly as of Sep. 7, 2020); or electronic Medicines Compendium (eMC) database (www.medicines.org.uk/emc/, in the version as of the priority date or the filing date of the present specification, particularly as of Sep. 7, 2020). Examples of diseases/disorders that are amenable to be treated or prevented with an analog or derivative of any particular peptide or protein drug include the same diseases/disorders that are amenable to be treated or prevented with the corresponding (underivatized) peptide or protein drug. Moreover, preferred examples of diseases/disorders that are amenable to be treated or prevented with any of the above-mentioned insulin or insulin analogs include, in particular, diabetes (e.g., type 1 diabetes mellitus or type 2 diabetes mellitus); preferred examples of diseases/disorders that are amenable to be treated or prevented with buserelin include, in particular, hormone-responsive cancer (such as, e.g., prostate cancer or breast cancer), or estrogen-dependent conditions (such as, e.g., endometriosis or uterine fibroids); buserelin can further be used, e.g., in assisted reproduction; preferred examples of diseases/disorders that are amenable to be treated or prevented with human growth hormone (hGH) or any of the above-mentioned hGH analogs or derivatives include, in particular, growth hormone deficiency; preferred examples of diseases/disorders that are amenable to be treated or prevented with fibroblast growth factor 21 (FGF21) include, in particular, cardiovascular disease, obesity, or diabetes (particularly type 2 diabetes); preferred examples of diseases/disorders that are amenable to be treated or prevented with any of the above-mentioned epoetin or any of its analogues or derivatives include, in particular, anemia, Alzheimer's disease (see, e.g., Maurice T et al., J Psychopharmacol. 2013; 27(11): 1044-57), Parkinson's disease (see, e.g., Alcalá-Barraza S R et al., J Drug Target. 2010; 18(3): 179-90), or multiple sclerosis; preferred examples of diseases/disorders that are amenable to be treated or prevented with the above-mentioned filgrastim or any derivatives thereof (e.g., PEG-filgrastim) include, in particular, low blood neutrophils due to a number of causes such as, e.g., chemotherapy, radiation poisoning, HIV or AIDS, or unknown causes; preferred examples of diseases/disorders that are amenable to be treated or prevented with the above-mentioned antibody adalimumab include, in particular, inflammatory or autoimmune diseases/disorders, and are more preferably selected from rheumatoid arthritis, psoriatic arthritis, ankylosing spondylitis, Crohn's disease, ulcerative colitis, psoriasis (e.g., chronic psoriasis), hidradenitis suppurativa, juvenile idiopathic arthritis, and birdshot retinochoroidopathy; preferred examples of diseases/disorders that are amenable to be treated or prevented with the above-mentioned antibody ustekinumab include, in particular, inflammatory or autoimmune diseases/disorders (e.g., intestinal inflammatory or intestinal autoimmune diseases/disorders), and are more preferably selected from Crohn's disease, ulcerative colitis, psoriasis (e.g., chronic psoriasis), and psoriatic arthritis.

The solid oral pharmaceutical composition according to the present invention can also be used to treat or prevent an intestinal disease/disorder, particularly an inflammatory, infectious or cancerous intestinal disease/disorder, such as, e.g., inflammatory bowel disease, Crohn's disease, ulcerative colitis, irritable bowel syndrome, a colonic bacterial infectious disease, or colorectal cancer. It will be understood that the peptide or protein drug comprised in the core of the solid oral pharmaceutical composition should in this case be a peptide or protein drug (particularly an antibody; e.g., adalimumab) effective against the respective intestinal disease/disorder.

The peptide or protein drug comprised in the solid oral pharmaceutical composition of the present invention may further be used in combination with one or more other therapeutic agents, particularly one or more other peptide or protein drugs (as described herein), which may be present in the solid oral pharmaceutical composition according to the invention, preferably in the core of the solid oral pharmaceutical composition, or may be provided in a separate pharmaceutical composition. In the latter case, the separate pharmaceutical composition may be administered simultaneously/concomitantly with the solid oral pharmaceutical composition according to the invention, or may be administered sequentially. It is preferred that any additional (other) peptide or protein drug is also provided in the core of the solid oral pharmaceutical composition according to the invention (such that all peptide or protein drugs are present in the core).

It will be understood that the peptide or protein drug (including any of the exemplary peptide or protein drugs described herein) may be present in non-salt form or in the form of a pharmaceutically acceptable salt. A corresponding pharmaceutically acceptable salt may be formed, e.g., by protonation of an atom carrying an electron lone pair which is susceptible to protonation, such as an amino group, with an inorganic or organic acid, or as a salt of a carboxylic acid group with a physiologically acceptable cation as they are well-known in the art. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts, nitrate salts, phosphate salts (such as, e.g., phosphate, hydrogenphosphate, or dihydrogenphosphate salts), carbonate salts, hydrogencarbonate salts or perchlorate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, glycolate, nicotinate, benzoate, salicylate, ascorbate, or pamoate (embonate) salts; sulfonate salts such as methanesulfonate (mesylate), ethanesulfonate (esylate), 2-hydroxyethanesulfonate (isethionate), benzenesulfonate (besylate), β-toluenesulfonate (tosylate), 2-naphthalenesulfonate (napsylate), 3-phenylsulfonate, or camphorsulfonate salts; and acidic amino acid salts such as aspartate or glutamate salts.

The solid oral pharmaceutical composition according to the present invention may also contain one or more amino acids, particularly arginine (preferably L-arginine) and/or lysine (preferably L-lysine). Arginine may be employed, e.g., in the form of the free base (in non-salt form) or in the form of a hydrochloride (HCl) salt. Lysine may likewise be employed, e.g., in the form of the free base (in non-salt form) or in the form of a hydrochloride salt. The amino acid(s), particularly arginine (e.g., L-arginine free base or L-arginine HCl) and/or lysine (e.g., L-lysine free base or L-lysine HCl), can be provided in the core of the solid oral pharmaceutical composition (together with the peptide or protein drug), which is particularly advantageous if the peptide or protein drug is an antibody. Thus, if the peptide or protein drug is an antibody (e.g., any of the exemplary/specific antibodies described herein above), it is preferred that the antibody is present (in the core of the solid oral pharmaceutical composition) in combination with one or more amino acids, more preferably in combination with arginine (e.g., L-arginine free base or L-arginine HCl) and/or lysine (e.g., L-lysine free base or L-lysine HCl).

The solid oral pharmaceutical composition may optionally comprise one or more further pharmaceutically acceptable excipients, such as carriers, diluents, fillers, disintegrants, lubricating agents, binders, colorants, pigments, stabilizers, preservatives, antioxidants, amino acids, reducing agents, bioadhesive agents and/or solubility enhancers. In particular, it may comprise one or more additives selected from vitamin E, histidine, microcrystalline cellulose (MCC), mannitol, starch, sorbitol and/or lactose. The solid oral pharmaceutical composition can be formulated by techniques known to the person skilled in the art, such as the techniques published in Remington's Pharmaceutical Sciences, 20^{th} Edition.

As noted above, the solid oral pharmaceutical composition may comprise one or more solubility enhancers, such as, e.g., poly(ethylene glycol), including poly(ethylene glycol) having a molecular weight in the range of about 200 to about 5,000 Da, ethylene glycol, propylene glycol, non-ionic surfactants, tyloxapol, polysorbate 20, polysorbate 80, macrogol-15-hydroxystearate, phospholipids, lecithin, dimyristoyl phosphatidylcholine, dipalmitoyl phosphatidylcholine, distearoyl phosphatidylcholine, cyclodextrins, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioethers, hydroxypropyl methylcellulose, hydroxypropylcellulose, polyvinylpyrrolidone, vinyl acetate copolymers, vinyl pyrrolidone, sodium lauryl sulfate, dioctyl sodium sulfosuccinate, or any combination thereof.

Moreover, as noted above, the solid oral pharmaceutical composition may comprise one or more pharmaceutically acceptable carriers. The pharmaceutically acceptable carrier may be an aqueous or non-aqueous agent, for example alcoholic or oleaginous, or a mixture thereof, and may contain a surfactant, an emollient, a lubricant, a stabilizer, a dye, a perfume, a preservative, an acid or base for adjustment of pH, a solvent, an emulsifier, a gelling agent, a moisturizer, a stabilizer, a wetting agent, a time release agent, a humectant, or any other component commonly included in a particular form of solid oral pharmaceutical composition. Pharmaceutically acceptable carriers are well known in the art and include, for example, aqueous solutions such as water or physiologically buffered saline or other solvents or vehicles such as glycols, glycerol, and oils such as olive oil or injectable organic esters. A pharmaceutically acceptable carrier can contain physiologically acceptable compounds that act, for example, to stabilize or to increase the absorption of the peptide or protein drug, for example, carbohydrates, such as glucose, sucrose or dextrans, antioxidants, such as ascorbic acid or glutathione, chelating agents, low molecular weight proteins or other stabilizers or excipients. A pharmaceutically acceptable carrier can also be selected from substances such as distilled water, benzyl alcohol, lactose, starches, talc, magnesium stearate, polyvinylpyrrolidone, alginic acid, colloidal silica, titanium dioxide, and flavoring agents. Preferred pharmaceutically acceptable carriers, in particular for use in the core of the solid oral pharmaceutical composition of the present invention, are selected from microcrystalline cellulose, mannitol, starch, sorbitol and lactose. Another preferred pharmaceutically acceptable carrier is magnesium stearate.

### The Core of the Solid Oral Pharmaceutical Composition

The core of the solid oral pharmaceutical composition of the present invention comprises a peptide or protein drug (as described above).

It is preferred that the core of the solid oral pharmaceutical composition further comprises a permeation enhancer (also referred to as a “mucosal permeation enhancer”). The use of a permeation enhancer improves or facilitates the mucosal absorption/permeation of the peptide or protein drug, particularly through the intestinal mucosa, and is advantageous particularly if the peptide or protein drug is a large molecule, e.g., a peptide or protein drug having a molecular weight of about 1 kDa or more.

The permeation enhancer may be, e.g., a zwitter-ionic permeation enhancer, a cationic permeation enhancer, an anionic permeation enhancer (e.g., an anionic permeation enhancer comprising one or more sulfonic acid groups (—SO₃H)), or a non-ionic permeation enhancer.

It is preferred that the permeation enhancer is selected from C₈₋₂₀ alkanoyl carnitine (preferably lauroyl carnitine, myristoyl carnitine or palmitoyl carnitine; e.g., lauroyl carnitine chloride, myristoyl carnitine chloride or palmitoyl carnitine chloride), salicylic acid (preferably a salicylate, e.g., sodium salicylate), a salicylic acid derivative (such as, e.g., 3-methoxysalicylic acid, 5-methoxysalicylic acid, or homovanillic acid, a C₈₋₂₀ alkanoic acid (preferably a C₈₋₂₀ alkanoate, more preferably a caprate, a caprylate, a myristate, a palmitate, or a stearate, such as, e.g., sodium caprate, sodium caprylate, sodium myristate, sodium palmitate, or sodium stearate), citric acid (preferably a citrate, e.g., sodium citrate), tartaric acid (preferably a tartrate), a fatty acid acylated amino acid (e.g., any of the fatty acid acylated amino acids described in US 2014/0056953 A1 which is incorporated herein by reference, including, without being limited thereto, sodium lauroyl alaninate, N-dodecanoyl-L-alanine, sodium lauroyl asparaginate, N-dodecanoyl-L-asparagine, sodium lauroyl aspartic acid, N-dodecanoyl-L-aspartic acid, sodium lauroyl cysteinate, N-dodecanoyl-L-cysteine, sodium lauroyl glutamic acid, N-dodecanoyl-L-glutamic acid, sodium lauroyl glutaminate, N-dodecanoyl-L-glutamine, sodium lauroyl glycinate, N-dodecanoyl-L-glycine, sodium lauroyl histidinate, N-dodecanoyl-L-histidine, sodium lauroyl isoleucinate, N-dodecanoyl-L-isoleucine, sodium lauroyl leucinate, N-dodecanoyl-L-leucine, sodium lauroyl methioninate, N-dodecanoyl-L-methionine, sodium lauroyl phenylalaninate, N-dodecanoyl-L-phenylalanine, sodium lauroyl prolinate, N-dodecanoyl-L-proline, sodium lauroyl serinate, N-dodecanoyl-L-serine, sodium lauroyl threoninate, N-dodecanoyl-L-threonine, sodium lauroyl tryptophanate, N-dodecanoyl-L-tryptophane, sodium lauroyl tyrosinate, N-dodecanoyl-L-tyrosine, sodium lauroyl valinate, N-dodecanoyl-L-valine, sodium lauroyl sarcosinate, N-dodecanoyl-L-sarcosine, sodium capric alaninate, N-decanoyl-L-alanine, sodium capric asparaginate, N-decanoyl-L-asparagine, sodium capric aspartic acid, N-decanoyl-L-aspartic acid, sodium capric cysteinate, N-decanoyl-L-cysteine, sodium capric glutamic acid, N-decanoyl-L-glutamic acid, sodium capric glutaminate, N-decanoyl-L-glutamine, sodium capric glycinate, N-decanoyl-L-glycine, sodium capric histidinate, N-decanoyl-L-histidine, sodium capric isoleucinate, N-decanoyl-L-isoleucine, sodium capric leucinate, N-decanoyl-L-leucine, sodium capric methioninate, N-decanoyl-L-methionine, sodium capric phenylalaninate, N-decanoyl-L-phenylalanine, sodium capric prolinate, N-decanoyl-L-proline, sodium capric serinate, N-decanoyl-L-serine, sodium capric threoninate, N-decanoyl-L-threonine, sodium capric tryptophanate, N-decanoyl-L-tryptophane, sodium capric tyrosinate, N-decanoyl-L-tyrosine, sodium capric valinate, N-decanoyl-L-valine, sodium capric sarcosinate, N-decanoyl-L-sarcosine, sodium oleoyl sarcosinate, sodium N-decylleucine, sodium stearoyl glutamate (e.g., Amisoft HS-11 P), sodium myristoyl glutamate (e.g., Amisoft MS-11), sodium lauroyl glutamate (e.g., Amisoft LS-11), sodium cocoyl glutamate (e.g., Amisoft CS-11), sodium cocoyl glycinate (e.g., Amilite GCS-11), sodium N-decyl leucine, sodium cocoyl glycine, sodium cocoyl glutamate, sodium lauroyl alaninate, N-dodecanoyl-L-alanine, sodium lauroyl asparaginate, N-dodecanoyl-L-asparagine, sodium lauroyl aspartic acid, N-dodecanoyl-L-aspartic acid, sodium lauroyl cysteinate, N-dodecanoyl-L-cysteine, sodium lauroyl glutamic acid, N-dodecanoyl-L-glutamic acid, sodium lauroyl glutaminate, N-dodecanoyl-L-glutamine, sodium lauroyl glycinate, N-dodecanoyl-L-glycine, sodium lauroyl histidinate, N-dodecanoyl-L-histidine, sodium lauroyl isoleucinate, N-dodecanoyl-L-isoleucine, sodium lauroyl leucinate, N-dodecanoyl-L-leucine, sodium lauroyl methioninate, N-dodecanoyl-L-methionine, sodium lauroyl phenylalaninate, N-dodecanoyl-L-phenylalanine, sodium lauroyl prolinate, N-dodecanoyl-L-proline, sodium lauroyl serinate, N-dodecanoyl-L-serine, sodium lauroyl threoninate, N-dodecanoyl-L-threonine, sodium lauroyl tryptophanate, N-dodecanoyl-L-tryptophane, sodium lauroyl tyrosinate, N-dodecanoyl-L-tyrosine, sodium lauroyl valinate, N-dodecanoyl-L-valine, N-dodecanoyl-L-sarcosine, sodium capric alaninate, N-decanoyl-L-alanine, sodium capric asparaginate, N-decanoyl-L-asparagine, sodium capric aspartic acid, N-decanoyl-L-aspartic acid, Sodium capric cysteinate, N-decanoyl-L-cysteine, sodium capric glutamic acid, N-decanoyl-L-glutamic acid, sodium capric glutaminate, N-decanoyl-L-glutamine, sodium capric glycinate, N-decanoyl-L-glycine, sodium capric histidinate, N-decanoyl-L-histidine, sodium capric isoleucinate, N-decanoyl-L-isoleucine, sodium capric leucinate, N-decanoyl-L-leucine, leucine, sodium capric methioninate, N-decanoyl-L-methionine, sodium capric phenylalaninate, N-decanoyl-L-phenylalanine, sodium capric prolinate, N-decanoyl-L-proline, sodium capric serinate, N-decanoyl-L-serine, sodium capric threoninate, N-decanoyl-L-threonine, sodium capric tryptophanate, N-decanoyl-L-tryptophane, sodium capric tyrosinate, N-decanoyl-L-tyrosine, sodium capric valinate, N-decanoyl-L-valine, sodium capric sarcosinate, sodium oleoyl sarcosinate, and pharmaceutically acceptable salts of any of the aforementioned compounds; or, e.g., C₈₋₂₀ alkanoyl sarcosinate (e.g., a lauroyl sarcosinate, such as sodium lauroyl sarcosinate) or one of the 20 standard proteinogenic α-amino acids that is acylated with a C₈₋₂₀ alkanoic acid), an alkylsaccharide (e.g., a C₁₋₂₀ alkylsaccharide, such as, e.g., C₈₋₁₀ alkylpolysaccharide like Multitrope™ 1620-LQ-(MV); or, e.g., n-octyl-beta-D-glucopyranoside, n-dodecyl-beta-D-maltoside, n-tetradecyl-beta-D-maltoside, tridecyl-beta-D-maltoside, sucrose laurate, sucrose stearate, sucrose myristate, sucrose palmitate, sucrose cocoate, sucrose mono-dodecanoate, sucrose mono-tridecanoate, sucrose mono-tetradecanoate, a coco-glucoside, or any of the alkylsaccharides described in U.S. Pat. No. 5,661,130 or in WO 2012/112319 which are herein incorporated by reference), a cyclodextrine (e.g., α-cyclodextrin, β-cyclodextrin, γ-cyclodextrin, methyl-β-cyclodextrin, hydroxypropyl β-cyclodextrin, or sulfobutylether β-cyclodextrin), N-[8-(2-hydroxybenzoyl)amino]caprylic acid (preferably a N-[8-(2-hydroxybenzoyl)amino]caprylate, more preferably sodium N-[8-(2-hydroxybenzoyl)amino]caprylate, also referred to as “SNAC”), a N-[8-(2-hydroxybenzoyl)amino]caprylate derivative (preferably a sodium N-[8-(2-hydroxybenzoyl)amino]caprylate derivative), a thiomer (also referred to as a thiolated polymer; may be synthesized, e.g., by immobilization of sulfhydryl bearing ligands on a polymeric backbone of well-established polymers such as, e.g., polyacrylic acid, carboxymethylcellulose or chitosan; exemplary thiomers include the thiomers that are described in Laffleur F et al., Future Med Chem. 2012, 4(17):2205-16 (doi: 10.4155/fmc.12.165) which is incorporated herein by reference), a mucoadhesive polymer having a vitamin B partial structure (e.g., any of the mucoadhesive polymers described in U.S. Pat. No. 8,980,238 B2 which is incorporated herein by reference; including, in particular, any of the polymeric compounds as defined in any one of claims 1 to 3 of U.S. Pat. No. 8,980,238 B2), a calcium chelating compound (e.g., ethylenediaminetetraacetic acid (EDTA), ethylene glycol tetraacetic acid (EGTA), sodium citrate, or polyacrylic acid), cremophor EL (also referred to as “Kolliphor EL”; CAS no. 61791-12-6), chitosan, N,N,N-trimethyl chitosan, benzalkonium chloride, bestatin, cetylpyridinium chloride, cetyltrimethylammonium bromide, a C₂₋₂₀ alkanol (e.g., ethanol, decanol, lauryl alcohol, myristyl alcohol, or palmityl alcohol), a C₈₋₂₀ alkenol (e.g., oleyl alcohol), a C₈₋₂₀ alkenoic acid (e.g., oleic acid), dextran sulfate, diethyleneglycol monoethyl ether (transcutol), 1-dodecylazacyclo-heptan-2-one (Azone®), caprylocaproyl polyoxylglycerides (such as, e.g., caprylocaproyl polyoxyl-8 glycerides; available, e.g., as Labrasol® or ACCONON® MC8-2), ethyl caprylate, glyceryl monolaurate, lysophosphatidylcholine, menthol, a C₈₋₂₀ alkylamine, a C₈₋₂₀ alkenylamine (e.g., oleylamine), phosphatidylcholine, a poloxamer, polyethylene glycol monolaurate, polyoxyethylene, polypropylene glycol monolaurate, a polysorbate (e.g., polysorbate 20 or polysorbate 80), cholic acid (preferably a cholate, e.g., sodium chlolate), a deoxycholate (e.g., sodium deoxycholate), a chenodeoxycholate (e.g., sodium chenodeoxycholate), sodium glycocholate, sodium glycodeoxycholate, sodium lauryl sulfate (SDS), sodium decyl sulfate, sodium octyl sulfate, sodium laureth sulfate, N-lauryl sarcosinate, decyltrimethyl ammonium bromide, benzyldimethyl dodecyl ammonium chloride, myristyltrimethyl ammonium chloride, dodecyl pyridinium chloride, decyldimethyl ammonio propane sulfonate, myristyldimethyl ammonio propane sulfonate, palmityldimethyl ammonio propane sulfonate, ChemBetaine CAS, ChemBetaine Oleyl, Nonylphenoxypolyoxyethylene, polyoxyethylene sorbitan monolaurate, polyoxyethylene sorbitan monopalmitate, sorbitan monooleate, Triton X-100, hexanoic acid, heptanoic acid, methyl laurate, isopropyl myristate, isopropyl palmitate, methyl palmitate, diethyl sebacate, sodium oleate, urea, lauryl amine, caprolactam, methyl pyrrolidone, octyl pyrrolidone, methyl piperazine, phenyl piperazine, Carbopol 934P, glycyrrhetinic acid, bromelain, pinene oxide, limonene, cineole, octyl dodecanol, fenchone, menthone, trimethoxy propylene methyl benzene, a cell-penetrating peptide (e.g., Klaklak, polyarginine or oligoarginine (particularly octa-arginine), penetratin (particularly L-penetratin), a penetratin analog (particularly PenetraMax; see, e.g., EI-Sayed Khafagy et al., Eur J Pharm Biopharm. 2013; 85(3 Pt A):736-43), HIV-1 Tat, transportan, or any of the cell-penetrating peptides referred to in US 2012/0065124), macrogol-15-hydroxystearate (e.g., Solutol HS 15), CriticalSorb (see, e.g., Ilium L et al. J Control Release. 2012; 162(1):194-200), a taurocholate (e.g., sodium taurocholate), a taurodeoxycholate (e.g., sodium taurodeoxycholate), a sulfoxide (e.g., a (C₁₋₁₀ alkyl)-(C₁₋₁₀ alkyl)-sulfoxide, such as, e.g., decyl methyl sulfoxide, or dimethyl sulfoxide), cyclopentadecalactone, 8-(N-2-hydroxy-5-chloro-benzoyl)-amino-caprylic acid (also referred to as “5-CNAC”), N-(10-[2-hydroxybenzoyl]amino)decanoic acid (also referred to as “SNAD”), dodecyl-2-N,N-dimethylamino propionate (also referred to as “DDAIP”), D-α-tocopheryl polyethylene glycol-1000 succinate (also referred to as “TPGS”), arginine, and pharmaceutically acceptable salts of the aforementioned compounds. Mixtures of two or more permeation enhancers, including any of the above-described permeation enhancers, can also be used. Moreover, any of the chemical permeation enhancers described in Whitehead K et al. Pharm Res. 2008 June; 25(6):1412-9 (particularly any one of those described in Table I of this reference), any one of the modified amino acids disclosed in U.S. Pat. No. 5,866,536 (particularly any one of compounds I to CXXIII, as disclosed in U.S. Pat. No. 5,866,536 which is incorporated herein by reference, or a pharmaceutically acceptable salt or solvate thereof, such as a disodium salt, an ethanol solvate, or a hydrate of any one of these compounds), any one of the modified amino acids disclosed in U.S. Pat. No. 5,773,647 (particularly any one of compounds 1 to 193, as disclosed in U.S. Pat. No. 5,773,647 which is incorporated herein by reference, or a pharmaceutically acceptable salt or solvate thereof, such as a disodium salt, an ethanol solvate, or a hydrate of any one of these compounds), any of the nanoparticles described in WO 2011/133198, any of the polymer preparations described in US 2015/174076 and/or a hydrogel (e.g., as described in Torres-Lugo M et al. Biotechnol Prog. 2002; 18(3):612-6) can likewise be used as permeation enhancer. Moreover, a complex lipoidal dispersion (e.g., a combination of an insoluble surfactant or oil with a soluble surfactant, and optionally with water or a co-solvent) can also be used as permeation enhancer; corresponding exemplary permeation enhancers include, in particular, mixed micelles, reversed micelles, a self-emulsifying system (e.g., SEDDS, SMEDDS, or SNEDDS), a lipid dispersion, a course emulsion, or solid lipid nanoparticles (SLNs). Preferably, the permeation enhancer is selected from sodium caprylate, sodium caprate, sodium laurate, sucrose laurate, sucrose stearate, sodium stearate, EDTA, polyacrylic acid, and N-[8-(2-hydroxybenzoyl)amino]caprylate or a pharmaceutically acceptable salt thereof (particularly sodium N-[8-(2-hydroxybenzoyl)amino]caprylate). A particularly preferred permeation enhancer is N-[8-(2-hydroxybenzoyl)amino]caprylate or a pharmaceutically acceptable salt thereof, in particular sodium N-[8-(2-hydroxybenzoyl)amino]caprylate.

Further preferred permeation enhancers are alkyl polysaccharides, arginine or CriticalSorb® (Solutol® HS15). In particular, the permeation enhancer may an alkyl glycoside (or a combination of two or more alkyl glycosides) which may be selected from any of the alkyl glycosides described in the following.

Alkyl glycosides to be used as permeation enhancer in accordance with the present invention can be synthesized by known procedures, i.e., chemically, as described, e.g., in Rosevear et al., Biochemistry 19:4108-4115 (1980) or Koeltzow and Urfer, J. Am. Oil Chem. Soc., 61:1651-1655 (1984), U.S. Pat. No. 3,219,656 or U.S. Pat. No. 3,839,318 or enzymatically, as described, e.g., in Li et al., J. Biol. Chem., 266:10723-10726 (1991) or Gopalan et al., J. Biol. Chem. 267:9629-9638 (1992).

Alkyl glycosides to be used as permeation enhancer in the present invention can include, but are not limited to: alkyl glycosides, such as octyl-, nonyl-, decyl-, undecyl-, dodecyl-, tridecyl-, tetradecyl-, pentadecyl-, hexadecyl-, heptadecyl-, and octadecyl-α- or β-D-maltoside, -glucoside or -sucroside (which may be synthesized according to Koeltzow and Urfer; Anatrace Inc., Maumee, Ohio; Calbiochem, San Diego, Calif.; Fluka Chemie, Switzerland); alkyl thiomaltosides, such as heptyl-, octyl-, dodecyl-, tridecyl-, and tetradecyl-β-D-thiomaltoside (which may be synthesized according to Defaye, J. and Pederson, C., “Hydrogen Fluoride, Solvent and Reagent for Carbohydrate Conversion Technology” in Carbohydrates as Organic Raw Materials, 247-265 (F. W. Lichtenthaler, ed.) VCH Publishers, New York (1991); Ferenci, T., J. Bacteriol, 144:7-11 (1980)); alkyl thioglucosides, such as heptyl- or octyl 1-thio α- or β-D-glucopyranoside (Anatrace, Inc., Maumee, Ohio; see Saito, S. and Tsuchiya, T. Chem. Pharm. Bull. 33:503-508 (1985)); alkyl thiosucroses (which may be synthesized according to, for example, Binder, T. P. and Robyt, J. F., Carbohydr. Res. 140:9-20 (1985)); alkyl maltotriosides (which may be synthesized according to Koeltzow and Urfer); long chain aliphatic carbonic acid amides of sucrose β-amino-alkyl ethers (which may be synthesized according to Austrian Patent 382,381 (1987); Chem. Abstr., 108:114719 (1988) and Gruber and Greber pp. 95-116); derivatives of palatinose and isomaltamine linked by amide linkage to an alkyl chain (which may be synthesized according to Kunz, M., “Sucrose-based Hydrophilic Building Blocks as Intermediates for the Synthesis of Surfactants and Polymers” in Carbohydrates as Organic Raw Materials, 127-153); derivatives of isomaltamine linked by urea to an alkyl chain (which may be synthesized according to Kunz); long chain aliphatic carbonic acid ureides of sucrose β-amino-alkyl ethers (which may be synthesized according to Gruber and Greber, pp. 95-116); and long chain aliphatic carbonic acid amides of sucrose β-amino-alkyl ethers (which may be synthesized according to Austrian Patent 382,381 (1987), Chem. Abstr., 108:114719 (1988) and Gruber and Greber, pp. 95-116).

The permeation enhancer may also be selected from any of the enhancing agents referred to in U.S. Pat. No. 8,927,497, including in particular any of alkyl glycosides, any of the saccharide alkyl esters, and/or any of the mucosal delivery-enhancing agents described in this document.

Moreover, the permeation enhancer may also be a compound of the following formula (I):

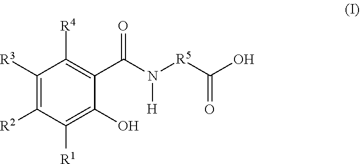

wherein:
R¹, R², R³ and R⁴ are each independently selected from hydrogen, —OH, —NR⁶R⁷, halogen (e.g., —F, —Cl, —Br or —I), C₁₋₄ alkyl or C₁₋₄ alkoxy;
R⁵ is a substituted or unsubstituted C₂₋₁₆ alkylene, substituted or unsubstituted C₂₋₁₆ alkenylene, substituted or unsubstituted C₁₋₁₂ alkyl(arylene) [e.g., substituted or unsubstituted C₁₋₁₂ alkyl(phenylene)], or substituted or unsubstituted aryl(C₁₋₁₂ alkylene) [e.g., substituted or unsubstituted phenyl(C₁₋₁₂ alkylene)]; and
R⁶ and R⁷ are each independently hydrogen, oxygen, —OH or C₁₋₄ alkyl;
or a pharmaceutically acceptable salt or solvate thereof, particularly a disodium salt, an alcohol solvate (e.g., a methanol solvate, an ethanol solvate, a propanol solvate, or a propylene glycol solvate, or any such solvate of the disodium salt; particularly an ethanol solvate or an ethanol solvate of the disodium salt), or a hydrate thereof (e.g., a monohydrate of the disodium salt). The above-mentioned “substituted” groups comprised in formula (I) are preferably substituted with one or more (e.g., one, two, or three) substituent groups independently selected from halogen (e.g., —F, —Cl, —Br or —I), —OH, C₁₋₄ alkyl or C₁₋₄ alkoxy. Such compounds and methods for their preparation are described, e.g., in WO 00/59863 which is incorporated herein by reference.

Accordingly, the permeation enhancer may also be a “delivery agent” as described in WO 00/59863. Preferred examples of the compounds of formula (I) include N-(5-chlorosalicyloyl)-8-aminocaprylic acid, N-(10-[2-hydroxybenzoyl]amino)decanoic acid, N-(8-[2-hydroxybenzoyl]amino)caprylic acid, a monosodium or disodium salt of any one of the aforementioned compounds, an ethanol solvate of the sodium salt (e.g., monosodium or disodium salt) of any one of the aforementioned compounds, a monohydrate of the sodium salt (e.g., monosodium or disodium salt) of any one of the aforementioned compounds, and any combination thereof. A particularly preferred compound of formula (I) is the disodium salt of N-(5-chlorosalicyloyl)-8-aminocaprylic acid or the monohydrate thereof.

It is particularly preferred that the permeation enhancer is selected from sodium caprate, sodium caprylate, mixtures of sodium caprate and sodium caprylate, SNAC, sucrose laurate, labrasol and polysorbate.

Furthermore, the permeation enhancer may also be a salt of a medium-chain fatty acid. The salt of a medium-chain fatty acid is preferably a salt of a C₄₋₁₈ saturated fatty acid, preferably a C₄₋₁₈ linear or branched alkanoic acid which optionally has 1, 2 or 3 C═C double bonds, more preferably a C₆₋₁₆ linear or branched alkanoic acid which optionally has 1, 2 or 3 C═C double bonds, even more preferably a C₆₋₁₄ linear or branched alkanoic acid which optionally has 1, 2 or 3 C═C double bonds. The salt of a medium-chain fatty acid is preferably a salt of a C₄₋₁₈ linear or branched alkanoic acid, more preferably a C₆₋₁₆ linear or branched alkanoic acid, even more preferably a C₆₋₁₄ linear or branched alkanoic acid. The salt of a medium-chain fatty acid is preferably selected from a salt of a valeric acid, caproic acid, enanthic acid, caprylic acid, pelargonic acid, capric acid, undecylic acid, lauric acid, tridecylic acid, myristic acid, pentadecylic acid, palmitic acid, margaric acid and/or stearic acid. More preferably, the salt of a medium-chain fatty acid is a salt of capric acid.

The salt of a medium-chain fatty acid is more preferably a sodium salt or a potassium salt. It is furthermore preferred that the salt of a medium-chain fatty acid is a salt of capric acid. Capric acid may also be referred to as decanoic acid (CH₃(CH₂)₈COOH). A preferred salt of capric acid is sodium caprate (i.e., CH₃(CH₂)₈COONa).

The solid oral pharmaceutical composition of the present invention preferably further comprises one or more enzyme inhibitors. Preferred enzyme inhibitors include trisodium phosphate (Na₃PO₄), arginine and lysine.

More preferably, the solid oral pharmaceutical composition of the present invention comprises a combination of sodium caprate and trisodium phosphate. Still more preferably, the core of the solid oral pharmaceutical composition of the present invention comprises sodium caprate, trisodium phosphate and the peptide or protein drug.

### Method of Preparing the Solid Oral Pharmaceutical Composition

The present invention furthermore relates to a method for preparing the solid oral pharmaceutical composition of the present invention. The method preferably comprises the steps of
preparing a core, and
applying the first coating completely surrounding the core.

Preferably, the first coating is applied using a first aqueous composition comprising a copolymer (A) in combination with a copolymer (B) and/or a copolymer (C) and/or a copolymer (D). More preferably, the first coating is applied using a first aqueous composition comprising a copolymer (A) in combination with a copolymer (B) and/or a copolymer (D).

The first aqueous composition preferably further comprises an anti-tacking agent. The anti-tacking agent is preferably selected from glycerol monostearate, talc or PlasAcryl.

In addition, the first aqueous composition preferably further comprises citric acid and/or has a pH in a range of 2 to 5, preferably 3 to 4.

Preferably, the method further comprises a step of applying the second coating completely surrounding the first coating, wherein the second coating is applied using a second aqueous composition comprising the copolymer (C) as defined above. The second aqueous composition optionally further comprises an anti-tacking agent, wherein the anti-tacking agent is preferably selected from glycerol monostearate and talc.

The method may further include applying any one or more of the other coatings described herein above, preferably by using an aqueous dispersion comprising the polymers and/copolymers and other intended components of the respective coating(s). It has been found that copolymers such as poly(methacrylic acid-co-methyl methacrylate) 1:2 (such as Eudragit S 100) may already dissolve at a pH of less than 7, in particular at a pH of about 6.5, when applied from an aqueous dispersion.

### Dosage Forms

The solid oral pharmaceutical composition of the present invention is preferably an oral dosage form, more preferably a peroral dosage form.

Accordingly, the solid oral pharmaceutical composition is preferably to be administered orally, particularly perorally (or is formulated for oral administration, particularly peroral administration). Even more preferably, the solid oral pharmaceutical composition is to be administered by oral ingestion, particularly by swallowing. The solid oral pharmaceutical composition can thus be administered to pass through the mouth into the gastrointestinal tract, which can also be referred to as “oral-gastrointestinal” administration.

In a preferred embodiment, the solid oral pharmaceutical composition is to be administered perorally (particularly by oral-gastrointestinal administration) to a subject/patient in fed state. Accordingly, it is preferred that the solid oral pharmaceutical composition is administered perorally after a meal, i.e. after the intake of food (e.g., within about 1 to 2 hours after a meal). In a further preferred embodiment, the solid oral pharmaceutical composition is to be administered perorally (particularly by oral-gastrointestinal administration) to a subject/patient after overnight fasting together with a meal. Thus, it is preferred that the solid oral pharmaceutical composition is administered perorally together with food (e.g., breakfast) in the morning, particularly after overnight fasting.

In particular, it is preferred that the solid oral pharmaceutical composition is in the form of a capsule or a tablet. The total weight of the solid oral pharmaceutical composition, such as the capsule or tablet, may be in the range of 100 mg to 1500 mg. The total weight of the solid oral pharmaceutical composition is more preferably in the range of 100 mg to 1200 mg, 200 mg to 1000 mg, 400 mg to 800 mg, or 600 mg to 900 mg. A tablet or capsule preferably has a total weight of at least 100 mg, such as 100 mg to 1200 mg, 400 mg to 800 mg or 600 mg to 900 mg.

Moreover, the core may be in the form of a multiparticulate. The term “multiparticulate” preferably refers to particles having a volume mean particle size, as determined by laser diffraction, of 0.05 to 2 mm. For example, the peptide or protein drug may be present in the form of a multitude of particles within a matrix of the permeation enhancer. More preferably, the core is in the form of a granulate or pellets.

Even more preferably, the solid oral pharmaceutical composition is formulated as a peroral dosage form for release of the peptide or protein drug in the small intestine and/or colon. Release in the ileum is more preferred. Coatings as set out in the present invention allow for a delayed release of the peptide or protein drug primarily in the lower part of the small intestine. In contrast to conventional solid oral pharmaceutical compositions based (mainly) on anionic polymers which dissolve at a pH of more than 7, the delayed release by the solid oral pharmaceutical compositions of the present invention is regulated by non-ionic pH-independent polymers. As a result, the small intestinal transit time is more constant and lower variations in drug release are observed, as compared to coatings mainly containing pH-dependent polymers. In addition, dose dumping, which can occur if a subject does not reach or reaches only for a short time a pH of more than 7, can be avoided by the solid oral pharmaceutical compositions of the present invention.

Furthermore, it is preferred that the solid oral pharmaceutical composition according to the invention has a dissolution profile (dissolution method according to the United States Pharmacopeia, USP) with less than 5% of the peptide or protein drug released within 1 hour at acidic stage (simulated gastric fluid according to USP or 0.1 M HCl), followed by dissolution in simulated intestinal fluid at pH between 6 and 6.5 with a lag time of at least 1 hour (more preferably at least 1.5 hours, even more preferably at least 2 hours, yet even more preferably at least 2.5 hours). Within the lag time, not more than 10% of the peptide or protein drug is released. After the lag time, more than 75% of the peptide or protein drug is released in simulated intestinal fluid at pH between 6 and 6.5 within 1 hour. Accordingly, it is preferred that the solid oral pharmaceutical composition has a dissolution profile, as determined by the dissolution method according to USP, with less than 5% of the Peptide or protein drug released within 1 hour in simulated gastric fluid, followed by dissolution in simulated intestinal fluid at pH between 6 and 6.5 with a lag time of at least 1 hour, whereby not more than 10% of the peptide or protein drug is released within the lag time, and whereby after the lag time more than 75% of the peptide or protein drug is released in simulated intestinal fluid at pH between 6 and 6.5 within 1 hour. As explained above, the dissolution profile should be determined in accordance with the US Pharmacopeia (USP, preferably in the version as of Sep. 1, 2020); alternatively, however, the dissolution profile may also be determined using the modified version of the USP dissolution method described herein below in Example 7, 18 or 19. Exemplary solid oral pharmaceutical compositions according to the present invention having such a dissolution profile are described below in the examples section.

It is furthermore preferred that the solid oral pharmaceutical composition according to the invention shows a release of less than 5% (preferably less than 3%, more preferably less than 1%, even more preferably no release) of the Peptide or protein drug in simulated fed state gastric media, such as e.g. FEDGAS pH 6 (see Example 18), for a period of at least 1 hour, preferably at least 2 hours, more preferably at least 3 hours, most preferably at least 4 hours.

Further preferred examples of solid oral pharmaceutical compositions according to the invention include compositions having a first coating comprising (or consisting of) HPMC, and a second coating (which is exterior to the first coating) comprising (or consisting of) Eudragit FL 30 D-55. In particular, it is preferred that the coating with Eudragit FL 30 D-55 has a thickness leading to a weight gain of at least 45% (w/w) relating to the weight of the first coating with HPMC (e.g., an empty HPMC capsule). Alternatively, it is preferred that the coating with Eudragit FL 30 D-55 has a thickness leading to a weight gain of at least 30 mg, more preferably at least 40 mg, even more preferably at least 50 mg, even more preferably at least 100 mg, per dosage form (e.g., capsule or tablet). Also preferred is a solid oral pharmaceutical composition in the form of a tablet, having a coating comprising (or consisting of) Eudragit FL 30 D-55 with a thickness leading to a weight gain of at least 10% (w/w) (relating to the weight of the tablet before coating with Eudragit FL 30 D-55).

Moreover, also preferred is a solid oral pharmaceutical composition according to the invention, which is in the form of a tablet, having a coating comprising Eudragit NM30D with a coating weight gain of at least 10% (w/w) (e.g., at least 15% (w/w), at least 20% (w/w), or at least 25% (w/w)) in relation to the weight of the uncoated tablet (i.e., before the application of the coating comprising Eudragit NM30D). Surprisingly, the inventors found that increasing the coating weight gain with coatings according to the present invention allows to further increase the oral bioavailability, especially under fed conditions.

Typically, a physician will determine the actual dosage of the peptide or protein drug which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy. The precise dose will ultimately be at the discretion of the attendant physician or veterinarian.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “treatment” of a disorder or disease as used herein is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease as used herein is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a peptide or protein drug according to the invention before any clinical and/or pathological symptoms are diagnosed or determined or can be diagnosed or determined by the attending physician.

The terms “peptide” and “protein”, as in the expression “peptide or protein drug”, are used herein interchangeably and refer to a polymer of two or more amino acids linked via amide bonds that are formed between an amino group of one amino acid and a carboxyl group of another amino acid. The amino acids comprised in the peptide or protein, which are also referred to as amino acid residues, may be selected from the 20 standard proteinogenic α-amino acids (i.e., Ala, Arg, Asn, Asp, Cys, Glu, Gln, Gly, His, lie, Leu, Lys, Met, Phe, Pro, Ser, Thr, Trp, Tyr, and Val) but also from non-proteinogenic and/or non-standard α-amino acids (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group such as, e.g., cyclopentylalanine, cyclohexylalanine, phenylalanine, naphthylalanine, pyridylalanine, thienylalanine, cyclohexylglycine, or phenylglycine) as well as β-amino acids (e.g., β-alanine), γ-amino acids (e.g., γ-aminobutyric acid, isoglutamine, or statine) and 6-amino acids. Preferably, the amino acid residues comprised in the peptide or protein are selected from α-amino acids, more preferably from the 20 standard proteinogenic α-amino acids (which can be present as the L-isomer or the D-isomer, and are preferably all present as the L-isomer). The peptide or protein may be unmodified or may be modified, e.g., at its N-terminus, at its C-terminus and/or at a functional group in the side chain of any of its amino acid residues (particularly at the side chain functional group of one or more Lys, His, Ser, Thr, Tyr, Cys, Asp, Glu, and/or Arg residues). Such modifications may include, e.g., the attachment of any of the protecting groups described for the corresponding functional groups in: Wuts P G & Greene T W, Greene's protective groups in organic synthesis, John Wiley & Sons, 2006. Such modifications may also include the covalent attachment of one or more polyethylene glycol (PEG) chains (forming a PEGylated peptide or protein), the glycosylation and/or the acylation with one or more fatty acids (e.g., one or more C₈₋₃₀ alkanoic or alkenoic acids; forming a fatty acid acylated peptide or protein). Moreover, such modified peptides or proteins may also include peptidomimetics, provided that they contain at least two amino acids that are linked via an amide bond (formed between an amino group of one amino acid and a carboxyl group of another amino acid). The amino acid residues comprised in the peptide or protein may, e.g., be present as a linear molecular chain (forming a linear peptide or protein) or may form one or more rings (corresponding to a cyclic peptide or protein). The peptide or protein may also form oligomers consisting of two or more identical or different molecules.

The term “amino acid” refers, in particular, to any one of the 20 standard proteinogenic α-amino acids (i.e., Ala, Arg, Asn, Asp, Cys, Glu, Gln, Gly, His, lie, Leu, Lys, Met, Phe, Pro, Ser, Thr, Trp, Tyr, and Val) but also to non-proteinogenic and/or non-standard α-amino acids (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group such as, e.g., cyclopentylalanine, cyclohexylalanine, phenylalanine, naphthylalanine, pyridylalanine, thienylalanine, cyclohexylglycine, or phenylglycine) as well as β-amino acids (e.g., β-alanine), γ-amino acids (e.g., γ-aminobutyric acid, isoglutamine, or statine) and/or 6-amino acids as well as any other compound comprising at least one carboxylic acid group and at least one amino group. Unless defined otherwise, an “amino acid” preferably refers to an α-amino acid, more preferably to any one of the 20 standard proteinogenic α-amino acids (which can be present as the L-isomer or the D-isomer, and are preferably present as the L-isomer).

The term “antibody” refers to any immunoglobulin molecule that specifically binds to (or is immunologically reactive with) a particular antigen. The antibody may be, e.g., a monoclonal antibody or a polyclonal antibody, and is preferably a monoclonal antibody. Moreover, the antibody (e.g., the monoclonal antibody) may be a whole antibody (e.g., IgA, IgD, IgE, IgM or IgG, including in particular IgG1, IgG2, IgG3 or IgG4), a chimeric antibody, a humanized antibody, a human antibody, a heteroconjugate antibody (e.g., a bispecific antibody), or it may be an antigen-binding fragment of any of the aforementioned types of antibody (such as, e.g., Fab, Fab′, F(ab′)₂, Fv, or scFv). The antibody may also be a single-chain antibody (scAb) or a single-domain antibody (sdAb; e.g., a “nanobody”).

The term “dissolves” as used herein preferably refers to a state in which the layer whose dissolution is to be determined has been sufficiently dissolved by a solution having the specified pH-value such that it becomes permeable (in particular for the peptide or protein drug, such as teduglutide). Whether a given layer dissolves, can be determined by using a dissolution apparatus 1 as described in United States Pharmacopeia (USP) General Chapter <711> Dissolution.

The term “surrounding” is used herein synonymously with “covering” or “completely covering”.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, if a component of a composition is indicated to be “optional”, the invention specifically relates to both possibilities, i.e., that the corresponding component is present (contained in the composition) or that the corresponding component is absent from the composition.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

As used herein, the term “about” refers to ±10% of the indicated numerical value, preferably to ±5% of the indicated numerical value, and in particular to the exact numerical value indicated.

Unless specifically indicated otherwise, all properties and parameters referred to herein (including, e.g., any amounts/concentrations indicated in “mg/ml” or in “% (v/v)”, and any pH values) are preferably to be determined at standard ambient temperature and pressure conditions, particularly at a temperature of 25° C. (298.15K) and at an absolute pressure of 101.325 kPa (1 atm).

Furthermore, it is to be understood that the present invention specifically relates to each and every combination of features and embodiments described herein, including any combination of general and/or preferred features/embodiments. In particular, the invention specifically relates to all combinations of preferred features described herein.

In this specification, a number of documents including patents, patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

In accordance with the detailed description above, the present invention particularly relates to the following items:
1. A solid oral pharmaceutical composition comprising:
   (i) a core comprising a peptide or protein drug, and
   (ii) a first coating, wherein the first coating comprises
      (ii-1) a copolymer (A) in combination with
      (ii-2) a copolymer (B) and/or a copolymer (C) and/or a copolymer (D);
      wherein the copolymer (A) comprises:
         (a) 20 to 90 mol-% ethyl acrylate repeating units, and
         (b) 10 to 80 mol-% methyl methacrylate repeating units;
      wherein the copolymer (B), if present, comprises:
         (a) 25 to 75 mol-% methacrylic acid repeating units, and
         (b) 25 to 75 mol-% ethyl acrylate repeating units;
      wherein the copolymer (C), if present, comprises:
         (a) 25 to 60 mol-% methacrylic acid repeating units, and
         (b) 40 to 75 mol-% methyl methacrylate repeating units;
      wherein the copolymer (D), if present, comprises:
         (a) 5 to 20 mol-% methacrylic acid repeating units, and
         (b) 20 to 40 mol-% methyl methacrylate repeating units, and
         (c) 60 to 75 mol-% methyl acrylate repeating units.
2. The solid oral pharmaceutical composition according to item 1, wherein the first coating comprises
   (ii-1) a copolymer (A) in combination with
   (ii-2) a copolymer (B) and/or a copolymer (D).
3. The solid oral pharmaceutical composition according to item 1 or 2, wherein the copolymer (A) in the first coating comprises 60 to 75 mol-% ethyl acrylate repeating units, and 25 to 40 mol-% methyl methacrylate repeating units.
4. The solid oral pharmaceutical composition according to any one of items 1 to 3, wherein the copolymer (A) in the first coating comprises ethyl acrylate repeating units and methyl methacrylate repeating units in a molar ratio of 2:1.
5. The solid oral pharmaceutical composition according to any one of items 1 to 4, wherein the copolymer (A) in the first coating comprises not more than 3 mol-% methyl acrylate repeating units, preferably wherein the copolymer (A) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
6. The solid oral pharmaceutical composition according to any one of items 1 to 5, wherein the copolymer (A) in the first coating consists of ethyl acrylate repeating units and methyl methacrylate repeating units.
7. The solid oral pharmaceutical composition according to any one of items 1 to 6, wherein the copolymer (A) in the first coating is a neutral non-ionic copolymer.
8. The solid oral pharmaceutical composition according to item 1 or 2, wherein the copolymer (A) in the first coating further comprises 0.5 to 20 mol-%, preferably 1 to 15 mol-%, 2-(trimethylammonio)ethyl methacrylate chloride repeating units.
9. The solid oral pharmaceutical composition according to any one of items 1, 2 and 8, wherein the copolymer (A) in the first coating comprises 25 to 39 mol-% ethyl acrylate repeating units, 60 to 74 mol-% methyl methacrylate repeating units, and 1 to 15 mol-% 2-(trimethylammonio)ethyl methacrylate chloride repeating units.

10. The solid oral pharmaceutical composition according to any one of items 1, 2, 8 and 9, wherein the copolymer (A) in the first coating comprises ethyl acrylate repeating units, methyl methacrylate repeating units, and 2-(trimethylammonio)ethyl methacrylate chloride repeating units in a molar ratio of 1:2:0.1 or 1:2:0.2.

11. The solid oral pharmaceutical composition according to any one of items 1, 2 and 8 to 10, wherein the copolymer (A) in the first coating comprises not more than 3 mol-% methyl acrylate repeating units, preferably wherein the copolymer (A) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
12. The solid oral pharmaceutical composition according to any one of items 1, 2 and 8 to 11, wherein the copolymer (A) in the first coating consists of ethyl acrylate repeating units, methyl methacrylate repeating units, and 2-(trimethylammonio)ethyl methacrylate chloride repeating units.
13. The solid oral pharmaceutical composition according to any one of items 1, 2 and 8 to 12, wherein the copolymer (A) in the first coating is a cationic copolymer.
14. The solid oral pharmaceutical composition according to any one of items 1 to 13, wherein the copolymer (B) in the first coating comprises 45 to 55 mol-% methacrylic acid repeating units, and 45 to 55 mol-% ethyl acrylate repeating units.
15. The solid oral pharmaceutical composition according to any one of items 1 to 14, wherein the copolymer (B) in the first coating comprises methacrylic acid repeating units and ethyl acrylate repeating units in a molar ratio of 1:1.
16. The solid oral pharmaceutical composition according to any one of items 1 to 15, wherein the copolymer (B) in the first coating comprises not more than 3 mol-% methyl acrylate repeating units, preferably wherein the copolymer (B) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
17. The solid oral pharmaceutical composition according to any one of items 1 to 16, wherein the copolymer (B) in the first coating consists of methacrylic acid repeating units and ethyl acrylate repeating units.
18. The solid oral pharmaceutical composition according to any one of items 1 to 17, wherein the copolymer (C) in the first coating comprises 45 to 55 mol-% methacrylic acid repeating units, and 45 to 55 mol-% methyl methacrylate repeating units.
19. The solid oral pharmaceutical composition according to any one of items 1 to 18, wherein the copolymer (C) in the first coating comprises methacrylic acid repeating units and methyl methacrylate repeating units in a molar ratio of 1:1.
20. The solid oral pharmaceutical composition according to any one of items 1 to 19, wherein the copolymer (C) in the first coating comprises not more than 3 mol-% methyl acrylate repeating units, preferably wherein the copolymer (C) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
21. The solid oral pharmaceutical composition according to any one of items 1 to 20, wherein the copolymer (C) in the first coating consists of methacrylic acid repeating units and methyl methacrylate repeating units.
22. The solid oral pharmaceutical composition according to any one of items 1 to 17, wherein the copolymer (C) in the first coating comprises 25 to 40 mol-% methacrylic acid repeating units, and 60 to 75 mol-% methyl methacrylate repeating units.
23. The solid oral pharmaceutical composition according to any one of items 1 to 17 and 22, wherein the copolymer (C) in the first coating comprises methacrylic acid repeating units and methyl methacrylate repeating units in a molar ratio of 1:2.
24. The solid oral pharmaceutical composition according to any one of items 1 to 17, 22 and 23, wherein the copolymer (C) in the first coating comprises not more than 3 mol-% methyl acrylate repeating units, preferably wherein the copolymer (C) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
25. The solid oral pharmaceutical composition according to any one of items 1 to 17 and 22 to 24, wherein the copolymer (C) in the first coating consists of methacrylic acid repeating units and methyl methacrylate repeating units.
26. The solid oral pharmaceutical composition according to any one of items 1 to 25, wherein the copolymer (C) in the first coating is obtained from an aqueous dispersion of copolymer (C).
27. The solid oral pharmaceutical composition according to any one of items 1 to 26, wherein the copolymer (D) in the first coating comprises 7 to 13 mol-% methacrylic acid repeating units, 25 to 31 mol-% methyl methacrylate repeating units, and 62 to 68 mol-% methyl acrylate repeating units.
28. The solid oral pharmaceutical composition according to any one of items 1 to 27, wherein the copolymer (D) in the first coating comprises methacrylic acid repeating units, methyl methacrylate repeating units, and methyl acrylate repeating units in a molar ratio of 1:3:7.
29. The solid oral pharmaceutical composition according to any one of items 1 to 28, wherein the copolymer (D) in the first coating consists of methacrylic acid repeating units, methyl methacrylate repeating units, and methyl acrylate repeating units.
30. The solid oral pharmaceutical composition according to any one of items 1 to 29, wherein the content of the copolymer (A) in the first coating is at least 25% (w/w), preferably at least 50% (w/w), more preferably at least 75% (w/w), even more preferably at least 80% (w/w), yet even more preferably at least 90% (w/w), in relation to the total weight of the first coating.
31. The solid oral pharmaceutical composition according to any one of items 1 to 30, wherein the first coating comprises the copolymer (A) and the copolymer (B), wherein the content of the copolymer (A) in the first coating is at least 25% (w/w), preferably at least 50% (w/w), more preferably at least 75% (w/w), even more preferably at least 80% (w/w), yet even more preferably at least 90% (w/w), in relation to the total weight of the copolymer (A) and the copolymer (B) in the first coating.
32. The solid oral pharmaceutical composition according to any one of items 1 to 30, wherein the first coating comprises the copolymer (A) and the copolymer (C), wherein the copolymer (C) is as defined in any one of items 18 to 21.
33. The solid oral pharmaceutical composition according to any one of items 1 to 30, wherein the first coating comprises the copolymer (A) and the copolymer (C), wherein the copolymer (C) is as defined in any one of items 22 to 26.
34. The solid oral pharmaceutical composition according to any one of items 1 to 33, wherein the first coating further comprises one or more polymers selected from ethyl cellulose, hydroxypropyl methylcellulose (HPMC), and polyvinyl acetate, either in addition to the copolymer (A) or instead of it.
35. The solid oral pharmaceutical composition according to any one of items 1, 3 to 26 and 30 to 34, wherein the first coating does not contain any copolymer (D) as defined in any one of items 1 or 27 to 29.
36. The solid oral pharmaceutical composition according to any one of items 1 to 35, wherein the first coating dissolves at a pH in the range of 5 to 7, preferably at a pH in the range of 5.5 to 6.5, more preferably at a pH in the range of 5.5 to 6.0.
37. The solid oral pharmaceutical composition according to any one of items 1 to 36, further comprising:
   (iii) a second coating which is exterior to the first coating, wherein the second coating comprises a copolymer (C);
      wherein the copolymer (C) comprises:
         (a) 25 to 60 mol-% methacrylic acid repeating units, and
         (b) 40 to 75 mol-% methyl methacrylate repeating units.
38. The solid oral pharmaceutical composition according to item 37, wherein the copolymer (C) in the second coating comprises 45 to 55 mol-% methacrylic acid repeating units, and 45 to 55 mol-% methyl methacrylate repeating units.
39. The solid oral pharmaceutical composition according to items 37 or 38, wherein the copolymer (C) in the second coating comprises methacrylic acid repeating units and methyl methacrylate repeating units in a molar ratio of 1:1.
40. The solid oral pharmaceutical composition according to any one of items 37 to 39, wherein the copolymer (C) in the second coating comprises not more than 3 mol-% methyl acrylate repeating units, preferably wherein the copolymer (C) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
41. The solid oral pharmaceutical composition according to item 37, wherein the copolymer (C) in the second coating consists of methacrylic acid repeating units and methyl methacrylate repeating units.
42. The solid oral pharmaceutical composition according to items 37 or 41, wherein the copolymer (C) in the second coating comprises 25 to 40 mol-% methacrylic acid repeating units, and 60 to 75 mol-% methyl methacrylate repeating units.
43. The solid oral pharmaceutical composition according to any one of items 37, 41 and 42, wherein the copolymer (C) in the second coating comprises methacrylic acid repeating units and methyl methacrylate repeating units in a molar ratio of 1:2.
44. The solid oral pharmaceutical composition according to any one of items 37 and 41 to 43, wherein the copolymer (C) in the second coating comprises not more than 3 mol-% methyl acrylate repeating units, preferably wherein the copolymer (C) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
45. The solid oral pharmaceutical composition according to any one of items 37 and 41 to 44, wherein the copolymer (C) in the second coating consists of methacrylic acid repeating units and methyl methacrylate repeating units.
46. The solid oral pharmaceutical composition according to any one of items 1 to 45, wherein the second coating dissolves at a pH in the range of 5 to 7, preferably at a pH in the range of 5.5 to 6.5, more preferably at a pH in the range of 5.5 to 6.0.
47. The solid oral pharmaceutical composition according to any one of items 1 to 46, wherein the peptide or protein drug has a molecular weight of equal to or less than about 300 kDa, preferably a molecular weight of equal to or less than about 150 kDa.
48. The solid oral pharmaceutical composition according to any one of items 1 to 47, wherein the peptide or protein drug is selected from insulin, an insulin analog, insulin lispro, insulin PEGlispro, insulin aspart, insulin glulisine, insulin glargine, insulin detemir, NPH insulin, insulin degludec, B29K(N(ε)hexadecanedioyl-γ-L-Glu) A14E B25H desB30 human insulin,
B29K(N(ε)octadecanedioyl-γ-L-Glu-OEG-OEG) desB30 human insulin,
B29K(N(ε)octadecanedioyl-γ-L-Glu) A14E B25H desB30 human insulin,
B29K(N(ε)eicosanedioyl-γ-L-Glu) A14E B25H desB30 human insulin,
B29K(N(ε)octadecanedioyl-γ-L-Glu-OEG-OEG) A14E B25H desB30 human insulin,
B29K(N(ε)eicosanedioyl-γ-L-Glu-OEG-OEG) A14E B25H desB30 human insulin,
B29K(N(ε)eicosanedioyl-γ-L-Glu-OEG-OEG) A14E B16H B25H desB30 human insulin,
B29K(N(ε)hexadecanedioyl-γ-L-Glu) A14E B16H B25H desB30 human insulin,
B29K(N(ε)eicosanedioyl-γ-L-Glu-OEG-OEG) A14E B16H B25H desB30 human insulin,
B29K(N(ε)octadecanedioyl) A14E B25H desB30 human insulin, GLP-2, a GLP-2 analog, a GLP-2 agonist, teduglutide, elsiglutide, glucose-dependent insulinotropic polypeptide, elamipretide, a cyclotide, recombinant factor VIIa, eptacog alfa, amylin, an amylin analog, pramlintide, a somatostatin analog, octreotide, lanreotide, pasireotide, goserelin, buserelin, leptin, a leptin analog, metreleptin, peptide YY, a peptide YY analog, glatiramer, leuprolide, desmopressin, a desmopressin analog, a vasopressin receptor 2 agonist peptide, osteocalcin, an osteocalcin analog or derivative, human growth hormone, a human growth hormone analog, a long-acting human growth hormone, fibroblast growth factor 21, somapacitan, hGH-CTP, an antibody, a glycopeptide antibiotic, a glycosylated cyclic or polycyclic nonribosomal peptide antibiotic, vancomycin, teicoplanin, telavancin, bleomycin, ramoplanin, decaplanin, a cyclotide, bortezomib, cosyntropin, chorionic gonadotropin, menotropin, sermorelin, luteinizing-hormone-releasing hormone, somatropin, calcitonin, calcitonin-salmon, pentagastrin, oxytocin, nesiritide, anakinra, enfuvirtide, pegvisomant, dornase alfa, lepirudin, anidulafungin, eptifibatide, interferon alfacon-1, interferon alpha-2a, interferon alpha-2b, interferon beta-1a, interferon beta-1b, interferon gamma-1b, peginterferon alfa-2a, peginterferon alfa-2b, peginterferon beta-1a, fibrinolysin, vasopressin, aldesleukin, an epoetin, epoetin alfa, darbepoetin alfa, epoetin beta, epoetin delta, epoetin omega, epoetin zeta, epoetin theta, methoxy polyethylene glycol-epoetin beta, continuous erythropoietin receptor activator, pegylated epo, albupoetin, an epo-dimer analogue, epo-Fc, carbamylated EPO, synthetic erythropoese protein, the low molecular epo analogue PBI-1402, filgrastim, PEG-filgrastim, interleukin-11, cyclosporine, glucagon, urokinase, viomycin, thyrotropin-releasing hormone, leucine-enkephalin, methionine-enkephalin, substance P, adrenocorticotropic hormone, parathyroid hormone, a parathyroid hormone fragment, teriparatide, PTH(1-31), PTH(2-34), parathyroid hormone-related protein, abaloparatide, linaclotide, carfilzomib, icatibant, ecallantide, cilengitide, a prostaglandin F2a receptor modulator, PDC31, abciximab, ranibizumab, alefacept, romiplostim, anakinra, abatacept, belatacept, and pharmaceutically acceptable salts thereof.
49. The solid oral pharmaceutical composition according to any one of items 1 to 47, wherein the peptide or protein drug is selected from GLP-2, a GLP-2 agonist, a GLP-2 analog, teduglutide, elsiglutide, insulin, human insulin, an insulin analog, insulin lispro, insulin PEGlispro, A14E B25H B29K(N(eps)octadecanedioyl-gGlu-OEG-OEG) desB30 human insulin, insulin aspart, insulin glulisine, insulin glargine, insulin detemir, NPH insulin, insulin degludec, an antibody, a somatostatin analog, octreotide, lanreotide, pasireotide, desmopressin, a desmopressin analog, a vasopressin receptor 2 agonist peptide, a parathyroid hormone fragment, teriparatide, PTH(1-31), and PTH(2-34).
50. The solid oral pharmaceutical composition according to any one of items 1 to 47, wherein the peptide or protein drug is selected from GLP-2, a GLP-2 agonist, a GLP-2 analog, teduglutide, elsiglutide, insulin, human insulin, an insulin analog, insulin lispro, insulin PEGlispro, A14E B25H B29K(N(eps)octadecanedioyl-gGlu-OEG-OEG) desB30 human insulin, insulin aspart, insulin glulisine, insulin glargine, insulin detemir, NPH insulin, insulin degludec, and an antibody.
51. The solid oral pharmaceutical composition according to any one of items 1 to 50, wherein the core further comprises one or more permeation enhancers.
52. The solid oral pharmaceutical composition according to any one of items 1 to 51, wherein the solid oral pharmaceutical composition is an oral dosage form.
53. The solid oral pharmaceutical composition according to any one of items 1 to 52, wherein the solid oral pharmaceutical composition is in the form of a capsule.
54. The solid oral pharmaceutical composition according to any one of items 1 to 52, wherein the solid oral pharmaceutical composition is in the form of a tablet.
55. The solid oral pharmaceutical composition according to any one of items 1 to 54, wherein the core is in the form of a multiparticulate, preferably wherein the core is in the form of a granulate or pellets.
56. The solid oral pharmaceutical composition according to any one of items 1 to 55 for use in therapy.
57. The solid oral pharmaceutical composition according to any one of items 1 to 55 for use in the treatment or prevention of a disease/disorder.
58. The solid oral pharmaceutical composition for use according to item 56 or 57, wherein the solid oral pharmaceutical composition is to be administered orally.
59. Use of the solid oral pharmaceutical composition according to any one of items 1 to 55 for the manufacture of a medicament for the treatment or prevention of a disease/disorder.
60. A method of treating or preventing a disease/disorder in a subject in need thereof, the method comprising orally administering a therapeutically effective amount of the solid oral pharmaceutical composition according to any one of items 1 to 55 to the subject.
61. A method of delivering a peptide or protein drug to a subject in need thereof, the method comprising orally administering the solid oral pharmaceutical composition according to any one of items 1 to 55 to the subject.

The present invention furthermore relates to a solid oral pharmaceutical composition comprising:
(i) a core comprising a peptide or protein drug (as described herein above), and
(ii) a first coating, wherein the first coating comprises Eudragit NM30D, wherein the first coating optionally further comprises a copolymer (B) and/or a copolymer (C) and/or a copolymer (D);
   wherein the copolymer (A) comprises:
      (a) 20 to 90 mol-% ethyl acrylate repeating units, and
      (b) 10 to 80 mol-% methyl methacrylate repeating units;
   wherein the copolymer (B), if present, comprises:
      (a) 25 to 75 mol-% methacrylic acid repeating units, and
      (b) 25 to 75 mol-% ethyl acrylate repeating units;
   wherein the copolymer (C), if present, comprises:
      (a) 25 to 60 mol-% methacrylic acid repeating units, and
      (b) 40 to 75 mol-% methyl methacrylate repeating units;
   wherein the copolymer (D), if present, comprises:
      (a) 5 to 20 mol-% methacrylic acid repeating units, and
      (b) 20 to 40 mol-% methyl methacrylate repeating units, and
      (c) 60 to 75 mol-% methyl acrylate repeating units.

The general and preferred features described herein above, including also the methods and uses described herein, analogously apply to the solid oral pharmaceutical composition described in this paragraph.

The invention will now be described by reference to the following examples which are merely illustrative and are not to be construed as a limitation of the scope of the present invention.

### EXAMPLES

### Materials

The following materials were used in the experimental examples: All Eudragit polymers and PlasAcryl were obtained from Evonik (Germany). All other chemicals were obtained from Sigma Aldrich (Austria) or VWR (Austria).

### Eudragit NM 30 D:

Aqueous dispersion of a neutral copolymer based on ethyl acrylate and methyl methacrylate. Chemical/IUPAC name: Poly(ethyl acrylate-co-methyl methacrylate) 2:1.

### Eudragit® L 30 D-55:

Aqueous dispersion of an anionic copolymer based on methacrylic acid and ethyl acrylate. The ratio of the free carboxyl groups to the ester groups is approximately 1:1.

### Eudragit S 100

Anionic copolymer based on methacrylic acid and methyl methacrylate; chemical name: Poly(methacrylic acid-co-methyl methacrylate) 1:2.

### Eudragit L 100

Anionic copolymer based on methacrylic acid and methyl methacrylate; chemical name: Poly(methacrylic acid-co-methyl methacrylate) 1:1.

### Eudragit FS30D

Anionic copolymer based on methyl acrylate, methyl methacrylate and methacrylic acid; chemical name: Poly(methyl acrylate-co-methyl methacrylate-co-methacrylic acid) 7:3:1

### Example 1: Coating of HPMC Capsules with a Combination of 80% Eudragit NM 30 D and 20% Eudragit L 30 D-55

Preparation of the Eudragit dispersions: The pH of 109 g of Eudragit NM 30 D was adjusted to about 3 with 1.3 ml of a 20% (w/w) citric acid solution. 67 g of distilled water were added under conventional stirring. 28 g of Eudragit L 30 D-55 were added followed by the addition of 2 g of Plasacryl T20. The final composition was stirred for 15 minutes with a conventional stirrer.

Coating of HPMC capsules: Empty size 1 HPMC capsules, each weighing about 70 mg, were coated with the Eudragit dispersion using a Glatt GC1 lab coater to a final weight of 95 mg, 119 mg and 125 mg.

### Example 2: Coating of HPMC Capsules with a Combination of 75% Eudragit NM 30 D and 25% Eudragit L 30 D-55

Preparation of the Eudragit dispersions: The pH of 100 g of Eudragit NM 30 D was adjusted to about 3 with 1.3 ml of a 20% (w/w) citric acid solution. 67 g of distilled water were added under conventional stirring. 33.3 g of Eudragit L 30 D-55 were then added, followed by the addition of 2 g of Plasacryl T20. The final composition was stirred for 15 minutes with a conventional stirrer.

Coating of HPMC capsules: Empty size 1 HPMC capsules, each weighing about 70 mg, were coated with the Eudragit dispersion using a Glatt GC1 lab coater to a final weight of 115 mg.

### Example 3: Eudragit L 100 Top-Coating on Coated Capsules from Example 2

Preparation of the dispersions: 20 g of Eudragit L 100 were dispersed in distilled 100 ml water. 11.2 ml of 1N NH₃ were added slowly under continuous stirring. Stirring was continued for 60 minutes. 10 g of triethylcitrate (TEC) were then added and stirring continued for another 60 minutes. Separately, 10 g of talc were homogenized in 50 g of water with a high shear mixer (Ultra Turrax). Both dispersions were combined under continuous stirring with a conventional stirrer.

Top-coating of capsules: Capsules from Example 2 (with a weight of 115 mg) were top-coated with the Eudragit L 100 dispersion obtained above using a Glatt GC1 lab coater to a final weight of 130 mg.

### Example 4: Eudragit S 100 Top-Coating on Coated Capsules from Example 2

Preparation of the dispersions: 19.9 g of Eudragit S 100 were dispersed in 100 ml distilled water. 13.5 ml of 1N NH₃ was added slowly under continuous stirring. Stirring was continued for 60 minutes. Then 10 g of Triethylcitrate (TEC) were added and stirring continued for another 60 minutes. Separately 10 g of talc were homogenized in 50 g of water with a high shear mixer (Ultra Turrax). Both dispersions were combined under continuous stirring with a conventional stirrer.

Top-coating of capsules: Capsules from Example 2 (with a weight of 115 mg) were top-coated with the Eudragit S 100 dispersion obtained above using a Glatt GC1 lab coater to a final weight of 132 mg.

### Example 5: Enteric Coating of HPMC Capsules with Eudragit S100 Redispersion Comprising 50% TEC

Preparation of the Eudragit aqueous dispersion: 99.4 g of EUDRAGIT® S 100 was added slowly into 500 ml of water and stirred for approx. 5 minutes with a conventional stirrer. 67.5 g of 1N NH₃ were then added slowly into the EUDRAGIT® suspension and stirred for about 60 minutes. Next 49.7 g of triethyl citrate (TEC) were added into the EUDRAGIT® suspension and stirred again for 60 minutes. 49.7 g of talc were homogenized in 233.7 g of water for 10 minutes with a high shear mixer (e.g. Ultra Turrax). Then the talc suspension was poured into the EUDRAGIT® dispersion while stirring with a conventional stirrer. Finally, the spray suspension was passed through a 0.5 mm sieve and stirred continuously.

Coating of HPMC capsules: HPMC capsules were coated with the Eudragit aqueous dispersion using a Glatt GC1 lab coater to a weight gain of 18%, 60% and 64% (calculated based on weight of empty capsules).

### Example 6: Enteric Coating of HPMC Capsules with Eudragit S100 Redispersion Comprising 70% TEC

Preparation of the Eudragit aqueous dispersion: 99.4 g of EUDRAGIT® S 100 was added slowly into 500 ml of water and stirred for approx. 5 minutes with a conventional stirrer. 67.5 g of 1N NH₃ were then slowly added into the EUDRAGIT® suspension and stirred for about 60 minutes. Next 70 g of triethyl citrate (TEC) were added into the EUDRAGIT® suspension and stirred for again for 60 minutes. 49.7 g of talc were homogenized in 233.7 g of water for 10 minutes with a high shear mixer (e.g. Ultra Turrax). Then the talc suspension was poured into the EUDRAGIT® dispersion while stirring with a conventional stirrer. Finally, the spray suspension was passed through a 0.5 mm sieve and stirred continuously.

Enteric coating of HPMC capsules: HPMC capsules were coated with the Eudragit aqueous dispersion using a Glatt GC1 lab coater to a weight gain of 30%, 50% and 63% (calculated based on weight of empty capsules).

### Example 7: Dissolution Testing of Coated Capsules

Pre-coated capsules as prepared in any of Examples 1 to 6 can be filled with a peptide or protein drug and sodium caprate. Dissolution studies are performed with an Erweka DT light 126, using the basket method at 37° C. and a rotation speed of 75 rpm. A modified version of the United States Pharmacopeia (USP) method is used. The capsules are put into the baskets and the baskets are placed in open blue cap bottles, containing 100 ml of the according dissolution media. The blue cap bottles are in direct contact with water, ensuring a core temperature inside the blue cap bottles of 37° C. (temperature is confirmed prior to starting the test with an external thermometer). Baskets are first placed into simulated gastric fluid according to USP for one hour, then for one hour in simulated intestinal fluid (SIF) according to USP with pH 6.0, then for one hour in SIF according to USP with pH 6.5 and finally in SIF according to USP with pH 6.8. Samples of 1 ml are withdrawn after 60 minutes and at further pre-determined time-points. 40 μl of the samples are injected into a HPLC system using a reversed phase gradient method (water/acetonitrile+0.1% trifluoroacetic acid; column: Waters Xselect CSH C18). Sampling is performed until full capsule dissolution, defined by a peptide or protein drug release of >75% of the theoretical release value. Following this procedure, it can be demonstrated that the capsules (or other oral dosage forms) coated in accordance with the present invention exhibit an advantageous dissolution profile, showing no drug release at the acid stage, followed by a lag time at intestinal pH levels and then a fast release of the peptide or protein drug.

### Example 8: Enteric Coating of Tablets Comprising Semaglutide and Sodium Caprate with 80% Eudragit NM30D and 20% Eudragit FS30D

Preparation of the tablets: A homogenous powder blend with 240 mg of semaglutide, 6 g of sodium caprate, 2.64 g of galenIQ 720, 1.56 g of Avicel PH-101 and 120 mg of magnesium stearate was prepared. Mixing was first done in a mortar followed by mixing with a Topitec powder blender. Aliquots of 880 mg were compressed into tablets with a Korsch EK0 single punch tablet press with an average compression force of approximately 15 kN.

Preparation of the Eudragit dispersion: The pH of 160 g of Eudragit NM 30 D was adjusted to a pH of about 3 with 2.0 ml of a 20% (w/w) citric acid solution. 100 g of distilled water were added under conventional stirring. 42 g of Eudragit FS30D was added followed by the addition of 3 g of Plasacryl T20. The final composition was stirred for 15 minutes with a conventional stirrer.

Coating of tablets: Tablets were blended with empty size 1 HPMC capsules and coated with the Eudragit dispersion obtained above using a Glatt GC1 lab coater to final tablet weight of about 934 mg which relates to a coating weight gain of about 54 mg per tablet or 6.1% (w/w) weight gain. This relates to about 42 mg of Eudragit NM30D (dry substance) per tablet.

### Example 9: In Vivo Food Interaction Study after Oral Administration of Semaglutide Tablets to Beagle Dogs

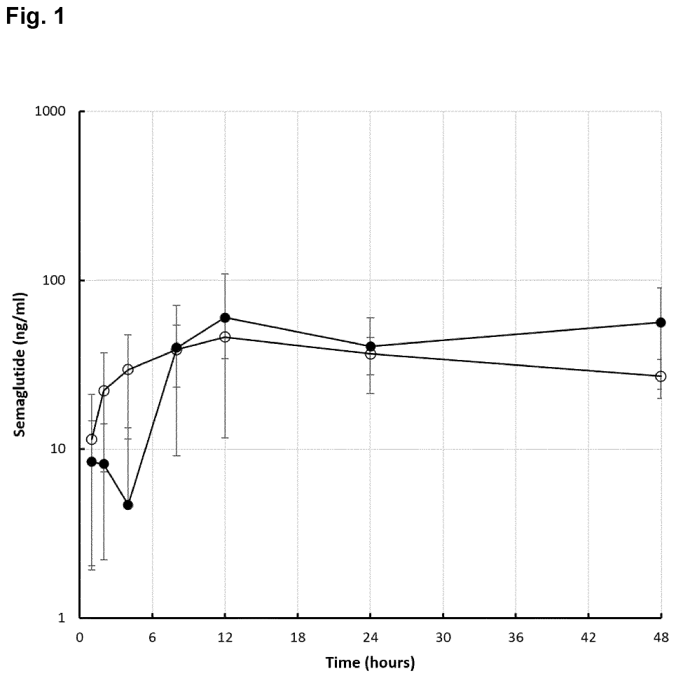

Food interaction was evaluated when tablets from Example 8 were administered orally with or without food as the presence of food within the stomach may interfere with the performance of orally administered peptide drugs. Animals were fasted overnight for at least 12 hours prior dose administration on day 0. Animals were offered food at 8-hour post-dose after sample collection. On the day of the experiment, intravenous catheter were placed on the saphenous or cephalic vein for blood sampling (20 G, 1-inch intravenous catheter). Tablets were administered perorally with 10 ml of water. Within 5 minutes after drug administration, dogs of the fed group received 50 g of a light meal (canine Hill's I/D), animals of the fasted group remained fasted for another 8 hours. Baseline blood samples were collected followed by oral administration of a single tablet per animal. Blood samples were collected at time points 0, 1, 2, 4, 8, 12, 24 and 48 into red top tubes and centrifuged for 10 min at 1,500 g and 4° C. Serum samples were duplicated and kept at −80° C. for further analysis by ELISA technique (Semaglutide Peninsula Laboratories International, Inc. Cat. No. S-1530). The results thus obtained are shown in FIG. 1.

Results: In the fed group, a delayed onset (avg. Tₘₐₓ) was observed in comparison to the fasted group. There was no statistical difference in the average AUC_{0-48 hrs} between the fasted and the fed group. The consumption of 50 grams of canine food within 5 minutes after tablet administration had no impact on the bioavailability of Semaglutide, compared to an animal cohort which did not receive any food. The tablet coating from Example 8 prevented a negative food impact.

### Example 10: Preparation of Tablets with Octreotide and Leuprolide

Preparation of the tablets: A homogenous powder blend with 260 mg of octreotide, 130 mg of leuprolide, 6.5 g of sodium caprate, 2.86 g of galenIQ 720, 1.69 g of Avicel PH-101 and 130 mg of magnesium stearate was prepared. Mixing was first done in a mortar followed by mixing with a Topitec powder blender. Aliquots of 890 mg were compressed into tablets with a Korsch EK0 single punch tablet press with an average compression force of approximately 15 kN.

### Example 11: Enteric Coating of Tablets Comprising Octreotide and Leuprolide with 80% Eudragit NM30D and 20% Eudragit L30 D-55

Preparation of the Eudragit dispersion: The pH of 107 g of Eudragit NM 30 D was adjusted to about 3 with 1.3 ml of a 20% (w/w) citric acid solution. 67 g of distilled water were added under conventional stirring. 28 g of Eudragit L 30 D-55 was added followed by the addition of 2 g of Plasacryl T20. The final composition was stirred for 15 minutes with a conventional stirrer.

Coating of tablets: Tablets from Example 10 were blended with placebo tablets of comparable weight and slightly different dimensions (to allow separation after the coating process) and coated with the Eudragit dispersion obtained above using a Glatt GC1 lab coater to final weight between of 954 mg which relates to a coating weight gain of 64 mg per tablet or 7.2% (w/w) weight gain. This relates to about 50 mg of Eudragit NM30D (dry substance) per tablet.

### Example 12: Enteric Coating of Tablets Comprising Octreotide and Leuprolide with Eudragit L30 D-55 (Reference Tablets)

Preparation of the Eudragit dispersions: 56.9 g of distilled water were added under conventional stirring to 114 g of Eudragit L 30 D-55, followed by the addition of 29.1 g of Plasacryl T20. The final composition was stirred for 15 minutes with a conventional stirrer.

Coating of tablets: Tablets from example 10 were coated with the Eudragit dispersion using a Glatt GC1 lab coater to a final weight of 956 mg (weight gain of 66 mg per tablet) which relates to a weight gain of 7.4% (w/w).

### Example 13: Preparation of Tablets with Human Insulin

Preparation of the tablets: A homogenous powder blend with 96 mg of human insulin, 6.0 g of sodium caprate, 2.64 g of galenIQ 720, 1.56 g of Avicel PH-101 and 120 mg of magnesium stearate was prepared. Mixing was first done in a mortar followed by mixing with a Topitec powder blender. Aliquots of 868 mg were compressed into tablets with a Korsch EK0 single punch tablet press with an average compression force of approximately 15 kN.

### Example 14: Enteric Coating of Tablets Comprising Human Insulin with 80% Eudragit NM30D and 20% Eudragit L30 D-55

Preparation of the Eudragit dispersion: The pH of 107 g of Eudragit NM 30 D was adjusted to about 3 with 1.3 ml of a 20% (w/w) citric acid solution. 67 g of distilled water were added under conventional stirring. 28 g of Eudragit L 30 D-55 was added followed by the addition of 2 g of Plasacryl T20. The final composition was stirred for 15 minutes with a conventional stirrer.

Coating of tablets: Tablets were blended with placebo tablets of comparable weight and slightly different dimensions (to allow separation after the coating process) and coated with the Eudragit dispersion obtained above using a Glatt GC1 lab coater to final weight of 913 mg which relates to a coating weight gain of 45 mg per tablet or 5.2% (w/w).

### Example 15: Enteric Coating of Tablets Comprising Human Insulin with Eudragit L30 D-55 (Reference Tablets)

Preparation of the Eudragit dispersions: 56.9 g of distilled water were added under conventional stirring to 114 g of Eudragit L 30 D-55, followed by the addition of 29.1 g of Plasacryl T20. The final composition was stirred for 15 minutes with a conventional stirrer.

Coating of tablets: Tablets from example 13 were coated with the Eudragit dispersion using a Glatt GC1 lab coater to a final weight of 916 mg (weight gain of 48 mg per tablet) which relates to a weight gain of 5.6% (w/w).

### Example 16: Enteric Coating of HPMC Capsules with Eudragit L 30 D-55 (Reference Capsules)

Preparation of the Eudragit dispersions: 56.9 g of distilled water were added under conventional stirring to 114 g of Eudragit L 30 D-55, followed by the addition of 29.1 g of Plasacryl T20. The final composition was stirred for 15 minutes with a conventional stirrer.

Coating of HPMC capsules and tablets: Empty size 1 HPMC capsules, each weighing about 70 mg were coated with the Eudragit dispersion using a Glatt GC1 lab coater to a final capsule weight of 104 mg (weight gain of 34 mg per capsule).

### Example 17: Enteric Coating of Capsules with 80% Eudragit NM30D and 20% Eudragit FS30D

Preparation of the Eudragit dispersion: The pH of 160.5 g of Eudragit NM 30 D was adjusted to about 3 with 2.0 ml of a 20% (w/w) citric acid solution. 100.5 g of distilled water were added under conventional stirring. 42 g of Eudragit FS30D was added followed by the addition of 3 g of Plasacryl T20. The final composition was stirred for 15 minutes with a conventional stirrer. Coating of capsules: Empty size 1 HPMC capsules were coated with the Eudragit dispersion obtained above using a Glatt GC1 lab coater to final capsule weight of about 102 mg which relates to a coating weight gain of about 32 mg per size 1 capsule.

### Example 18: Stability of Enteric Solid Dosage Forms Comprising Different Peptides in FEDGAS

The aim of this experiment is to demonstrate the robustness of coated solid dosage forms according to the invention in simulated fed state gastric media. Coated tablets from example 11, 12, 14, 15 and pre-coated capsules from examples 1, 16 and 17 were used and in case of capsules filled with 20 mg octreotide, 10 mg leuprolide, 8 mg insulin and 200 mg Sodium Caprate. Dissolution testing was done in FEDGAS (Fed State Simulated Gastric Fluid) at pH 6. FEDGAS™ pH 6 was prepared according to protocols from Biorelevant (www.biorelevant.com). For each 100 ml of final media, 4.1 ml of Biorelevant buffer pH 6, 81.4 g of water and 17 g of FEDGAS gel were mixed together. Dissolution studies were performed with an Erweka DT light 126, using the basket method at 37° C. and a rotation speed of 75 rpm. A modified version of the United States Pharmacopeia (USP) method was used. The capsules and tablets were put into the baskets and the baskets were placed in open blue cap bottles, containing 100 ml of the FEDGAS pH 6 media. The blue cap bottles were in direct contact with water, ensuring a core temperature inside the blue cap bottles of 37° C. (temperature was confirmed prior to starting the test with an external thermometer). Baskets were placed into FEDGAS pH 6 media for 6 hours. Samples of 1 ml were withdrawn after 60 minutes and at further pre-determined time-points. 40 μl of the samples were injected into a HPLC system using a reversed phase gradient method (water/acetonitrile+0.1% trifluoroacetic acid; column: Waters Xselect CSH C18).

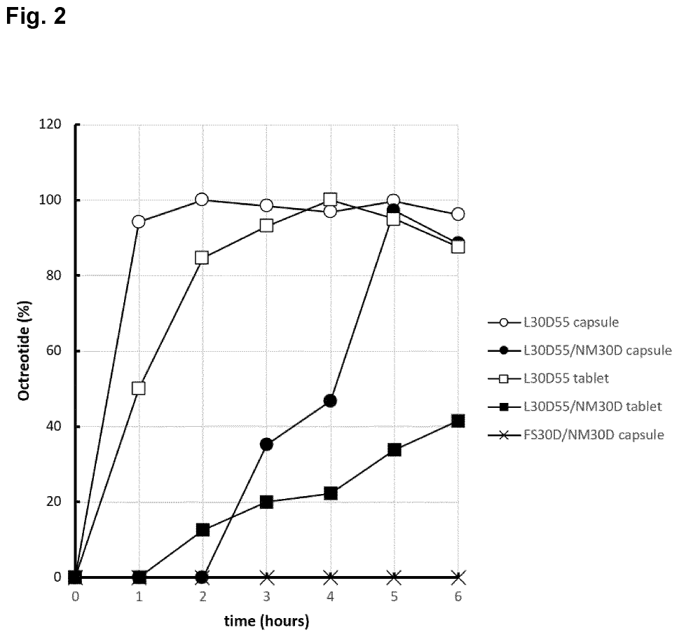

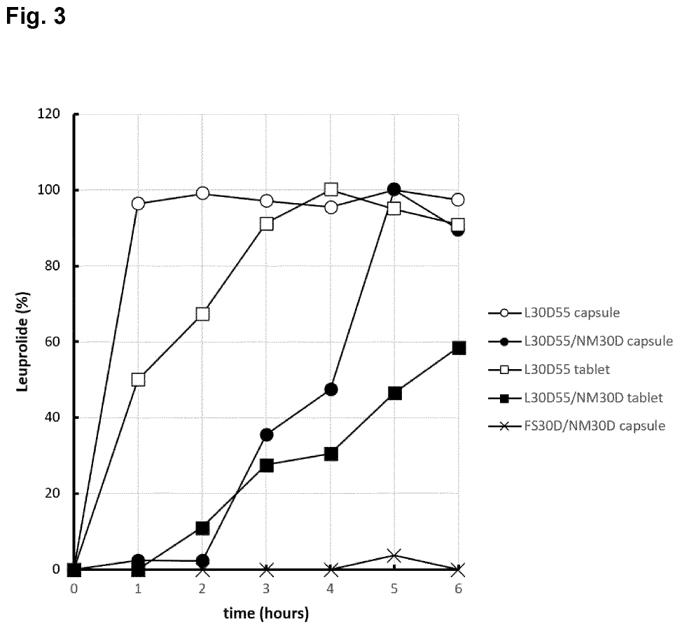

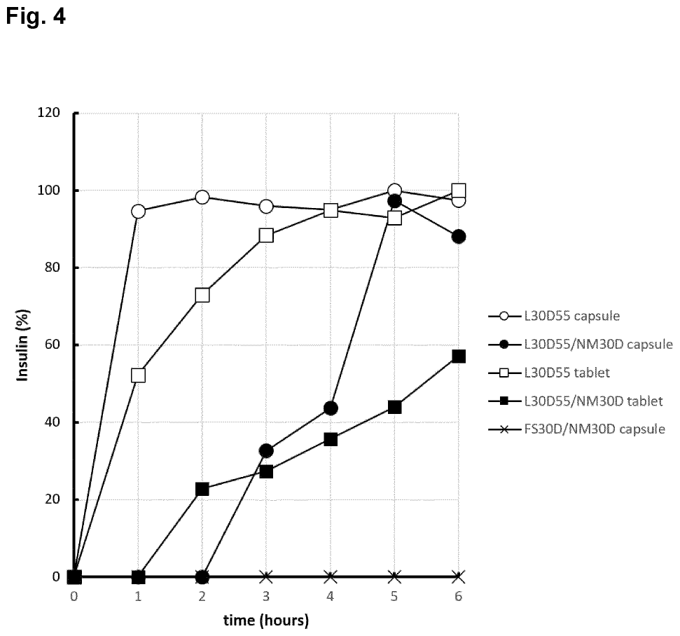

The results thus obtained are shown in FIGS. 2, 3 and 4. These results show that
1. Eudragit NM30D increases the robustness of enteric solid dosage forms comprising peptides in simulated fed state
2. Coated tablets according to the invention are more robust in simulated fed state than coated capsules
3. Coatings comprising the combination of Eudragit NM30D and Eudragit FS30D resulted in superior stability in simulated fed state compared with all other coatings.

### Example 19: Dissolution Testing of Coated Capsules

Pre-coated capsules from example 1 and 16 were filled each with 20 mg octreotide, 10 mg leuprolide, 8 mg insulin and 200 mg of sodium caprate. Dissolution studies were performed with an Erweka DT light 126, using the basket method at 37° C. and a rotation speed of 75 rpm. A modified version of the United States Pharmacopeia (USP) method was used. The capsules were put into the baskets and the baskets were placed in open blue cap bottles, containing 100 ml of the according dissolution media. The blue cap bottles were in direct contact with water, ensuring a core temperature inside the blue cap bottles of 37° C. (temperature was confirmed prior to starting the test with an external thermometer). Baskets were first placed into simulated gastric fluid according to USP for one hour, then for 3 hours in simulated intestinal fluid (SIF) according to USP with pH 6.0. Samples of 1 ml were withdrawn after 60 minutes and at further pre-determined time-points. 40 μl of the samples were injected into a HPLC system using a reversed phase gradient method (water/acetonitrile+0.1% trifluoroacetic acid; column: Waters Xselect CSH C18).

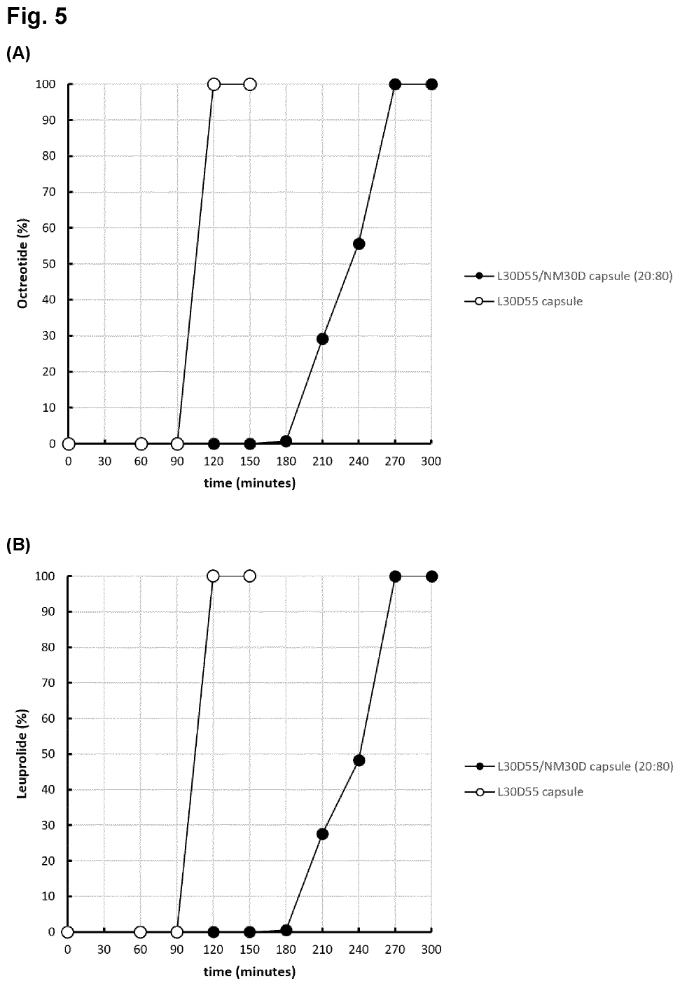

The results thus obtained are shown in FIGS. 5A, 5B and 5C. These results show that all capsules were stable at the acid stage but only capsules coated with significant amounts of Eudragit NM30D demonstrate afterwards a lag time at intestinal pH 6 levels followed by a rather fast release profile.

### Example 20: In Vivo Evaluation of Enteric Tablets Comprising Insulin and Sodium Caprate After Oral Administration to Cynomolgus Monkeys

Preparation of insulin tablets: A homogenous powder blend with 50 mg of human insulin, 1.160 g of sodium caprate, 510 mg of galenIQ 720, 300 mg of Avicel PH-101 and 20 mg of magnesium stearate was prepared. Mixing was first done in a mortar followed by mixing with a Topitec powder blender. Aliquots of 204 mg were compressed into tablets with a Korsch EK0 single punch tablet press with an average compression force of approximately 18 kN.

Enteric coating of tablets with 80% Eudragit NM30D and 20% Eudragit L30 D-55 (Sample 20a): Preparation of the Eudragit dispersion: The pH of 214 g of Eudragit NM 30 D was adjusted to about 3 with 2.6 ml of a 20% (w/w) citric acid solution. 134 g of distilled water were added under conventional stirring. 56 g of Eudragit L 30 D-55 was added followed by the addition of 4 g of Plasacryl T20. The final composition was stirred for 15 minutes with a conventional stirrer.

Coating of tablets: Tablets were coated with the Eudragit dispersion obtained above using a Glatt GC1 lab coater to final weight of 217.5 mg which relates to a coating weight gain of 6.5% (w/w).

Enteric coating of tablets comprising human Insulin with Eudragit L30 D-55 (reference tablets, Sample 20b): Preparation of the Eudragit dispersions: 85.5 g of distilled water were added under conventional stirring to 171 g of Eudragit L 30 D-55, followed by the addition of 43.5 g of Plasacryl T20. The final composition was stirred for 15 minutes with a conventional stirrer. Tablets were coated with the Eudragit dispersion using a Glatt GC1 lab coater to a final weight of 217.0 mg which relates to a weight gain of 6.5% (w/w).

Enteric insulin tablets as described above were dosed orally to overnight fasted female cynomolgus macaques with a body weight between 3.0 and 4.2 kg. Blood was collected at time points 0 (pre-dose), 1, 1.5, 2, 2.5, 3, 3.5 and 4 hours after oral administration of the reference tablet of Sample 20b. Blood was collected at time points 0 (pre-dose), 2, 2.5, 3, 3.5, 4, 4.5, 5, 5.5 and 6 hours after oral administration of the tablet of Sample 20a. Human insulin plasma concentrations were analysed with a commercial High Sensitivity Human Insulin ELISA kit from Abcam®. A summary of obtained pharmacokinetic parameters is shown in Table 1.

TABLE 1
Pharmacokinetic parameters after oral administration
of insulin tablets to Cynomolgus monkeys

| Formulation | Tₘₐₓ | AUC_{improvement factor} |
|---|---|---|
| Insulin tablet with sodium caprate | 1.2 hours | 1 |
| and Eudragit L30D-55 coating | | |
| (reference tablet) (Sample 20b) | | |
| Insulin tablet with sodium caprate | 4.0 hours | 13.3 |
| and Eudragit NM30D and Eudragit | | |
| L30D-55 coating (Sample 20a) | | |

### Example 21: In Vivo Evaluation of Enteric Tablets Comprising Leuprolide and Sodium Caprate after Oral Administration in Cynomolgus Monkeys

Preparation of leuprolide and octreotide tablets: A homogenous powder blend with 110 mg of octreotide, 55 mg Leuprolide, 1.210 g of sodium caprate, 550 mg of galenIQ 720, 330 mg of Avicel PH-101 and 55 mg of magnesium stearate was prepared. Mixing was first done in a mortar followed by mixing with a Topitec powder blender. Aliquots of 210 mg were compressed into tablets with a Korsch EK0 single punch tablet press with an average compression force of approximately 25 kN.

Enteric coating of tablets with 80% Eudragit NM30D and 20% Eudragit L30 D-55 (Sample 21a): Preparation of the Eudragit dispersion: The pH of 120 g of Eudragit NM 30 D was adjusted to about 3 with 1.5 ml of a 20% (w/w) citric acid solution. 75 g of distilled water were added under conventional stirring. 31.5 g of Eudragit L 30 D-55 was added followed by the addition of 2.25 g of Plasacryl T20. The final composition was stirred for 15 minutes with a conventional stirrer.

Coating of tablets: Tablets were coated with the Eudragit dispersion obtained above using a Glatt GC1 lab coater to final weight of 225 mg which relates to a coating weight gain of 7% (w/w).

Enteric Coating of Tablets Comprising Human Insulin with Eudragit L30 D-55 (Reference Tablets, Sample 21b):

Preparation of the Eudragit dispersions: 60 g of distilled water were added under conventional stirring to 120 g of Eudragit L 30 D-55, followed by the addition of 30 g of Plasacryl T20. The final composition was stirred for 15 minutes with a conventional stirrer. Tablets were coated with the Eudragit dispersion using a Glatt GC1 lab coater to a final weight of 221 mg which relates to a weight gain of 6% (w/w).

Enteric leuprolide/octreotide tablets were dosed orally to overnight fasted female cynomolgus macaques. Blood was collected at time points 0 (pre-dose), 30, 60, 90, 120, 150, 180 and 240 minutes after oral administration of reference tablet with Eudragit L30D-55 coating. Blood was collected at time points 0 (pre-dose), 120, 150, 180, 210, 240, 270, 300, 330 and 360 minutes after oral administration of tablet with 80% Eudragit NM30D and 20% Eudragit L30D-55 coating. Leuprolide plasma concentrations were analysed with a commercial leuprolide ELISA kit from BMA Biomedicals. A summary of obtained pharmacokinetic parameters is shown in Table 2.

TABLE 2
Pharmacokinetic parameters after oral administration
of leuprolide tablets to Cynomolgus monkeys

Improvement factor
vs. reference

| Formulation | Cₘₐₓ | AUC_{(0-4 hr)} |
|---|---|---|
| Leuprolide tablet with sodium caprate | 1.0 | 1.0 |
| and Eudragit L30D-55 coating (reference | | |
| tablet) (Sample 21b) | | |
| Leuprolide tablet with sodium caprate | 6.6 | 5.1 |
| and Eudragit NM30D and Eudragit | | |
| L30D-55 coating (Sample 21a) | | |

### Example 22: In Vivo Evaluation of Enteric Tablets Comprising Octreotide and Sodium Caprate after Oral Administration in Cynomolgus Monkeys

Enteric octreotide tablets from Example 21 were dosed orally to cynomolgus macaques. Blood was collected at time points 0 (pre-dose), 30, 60, 90, 120, 150, 180 and 240 minutes after oral administration of reference tablet with Eudragit L30D-55 coating (Sample 21b). Blood was collected at time points 0 (pre-dose), 120, 150, 180, 210, 240, 270, 300, 330 and 360 minutes after oral administration of tablet with 80% Eudragit NM30D and 20% Eudragit L30D-55 coating (Sample 21a). Octreotide plasma concentrations were analysed with a commercial octreotide ELISA kit from BMA Biomedicals. A summary of obtained pharmacokinetic parameters is shown in Table 3.

TABLE 3
Pharmacokinetic parameters after oral administration
of octreotide tablets to Cynomolgus monkeys

| | | | Improvement factor | |
|---|---|---|---|---|
| | | | vs. reference | |
| Formulation | Tₘₐₓ | | Cₘₐₓ | AUC |
| Octreotide tablet with sodium | 1 | hour | 1.0 | 1.0 |
| caprate and Eudragit L30D-55 | | | | |
| coating (reference tablet) | | | | |
| (Sample 21b) | | | | |
| Octreotide tablet with sodium | 2.5 | hours | 1.7 | 3.1 |
| caprate and Eudragit NM30D | | | | |
| and Eudragit L30D-55 coating | | | | |
| (Sample 21a) | | | | |

### Example 23: In Vivo Evaluation of Enteric Solid Oral Dosage Forms Comprising Semaglutide And Sodium Caprate after Gastroscopic Administration to the Duodenum of Pigs

Preparation of the tablets: Tablets with each 20 mg of Semaglutide, 500 mg sodium caprate, 220 mg sorbitol, 130 mg Avicel and 10 mg magnesium stearate were prepared.

### Coating of the Tablets:

Reference tablets B: Tablets were coated with Eudragit L30D-55
Coated tablets C: Tablets were coated with a mixture of 80% Eudragit NM30D and 20% Eudragit L30D-55
Coated tablets D: Tablets were coated with a mixture of 80% Eudragit NM30D and 20% Eudragit FS30D

Gastroscopic administration to pigs: The formulations were directly dosed into the duodenum of anaesthetized pigs with a gastroscope. Blood was taken at time points 0 prior dosing and at 1, 2, 4, 6, 8 and 24 hours after dosing. Plasma concentrations of the Semaglutide were analysed with LC-MS. The results are shown in Table 4. Only enteric tablets coated according to the invention resulted in significant plasma levels of Semaglutide.

TABLE 4
Pharmacokinetic parameters of Semaglutide after intestinal
administration of solid dosage forms in pigs

| | Tₘₐₓ | Cₘₐₓ |
|---|---|---|
| Formulations | (hrs.) | (ng/ml) |
| Semaglutide tablet with sodium caprate | 2.0 | <LoQ |
| uncoated (reference tablet A) | | |
| Semaglutide tablet with sodium caprate | 2.7 | <LoQ |
| coated with Eudragit L30D-55 (reference | | |
| tablet B) | | |
| Semaglutide tablet with sodium caprate | 7.3 | 150.7 |
| coated with 80% Eudragit NM30D and | | |
| 20% Eudragit L30D-55 (coated tablets C) | | |
| Semaglutide tablet with sodium caprate | 5.3 | 150.4 |
| coated with 80% Eudragit NM30D and | | |
| 20% Eudragit FS30D (coated tablets D) | | |

LoQ refers to the limit of quantitation.

### Example 24: Enteric Coating of Octreotide and Semaglutide Tablets with Eudragit L30D-55 (Reference Tablets)

Preparation of the tablets: A homogenous powder blend with 410 mg of semaglutide, 410 mg of octreotide, 10.25 g of sodium caprate, 4.1 g of sorbitol (Neosorb), 2.665 g of Avicel PH-101 and 205 mg of magnesium stearate was prepared. Mixing was first done in a mortar followed by mixing with a Topitec powder blender. Aliquots of 880 mg were compressed into tablets with a Korsch EK0 single punch tablet press with an average compression force of approximately 11 kN.

Preparation of the Eudragit dispersions: 58 g of distilled water were added under conventional stirring to 114 g of Eudragit L 30 D-55, followed by the addition of 29 g of Plasacryl T20. The final composition was stirred for 15 minutes with a conventional stirrer.

Coating of tablets: Tablets were coated with the Eudragit dispersion using a Glatt GC1 lab coater to a final weight of 931 mg (weight gain of 51 mg per tablet) which relates to a weight gain of 5.8% (w/w).

### Example 25: Enteric Coating of Octreotide and Semaglutide Tablets with 70% Eudragit NM30D and 30% Eudragit FS30D

Preparation of the tablets: A homogenous powder blend with 410 mg of semaglutide, 410 mg of octreotide, 10.25 g of sodium caprate, 4.1 g of sorbitol (Neosorb), 2.665 g of Avicel PH-101 and 205 mg of magnesium stearate was prepared. Mixing was first done in a mortar followed by mixing with a Topitec powder blender. Aliquots of 880 mg were compressed into tablets with a Korsch EK0 single punch tablet press with an average compression force of approximately 11 kN.

Preparation of the Eudragit dispersion: The pH of 140 g of Eudragit NM 30 D was adjusted to a pH of about 3 with 2.0 ml of a 20% (w/w) citric acid solution. 100 g of distilled water were added under conventional stirring. 60 g of Eudragit FS30D was added followed by the addition of 3 g of Plasacryl T20. The final composition was stirred for 15 minutes with a conventional stirrer.

Coating of tablets: Tablets were blended with empty size 1 HPMC capsules and coated with the Eudragit dispersion obtained above using a Glatt GC1 lab coater to final tablet weight of about 927 mg which relates to a coating weight gain of about 47 mg per tablet or 5.4% (w/w) weight gain.

### Example 26: Dissolution of Enteric Octreotide and Semaglutide Tablets

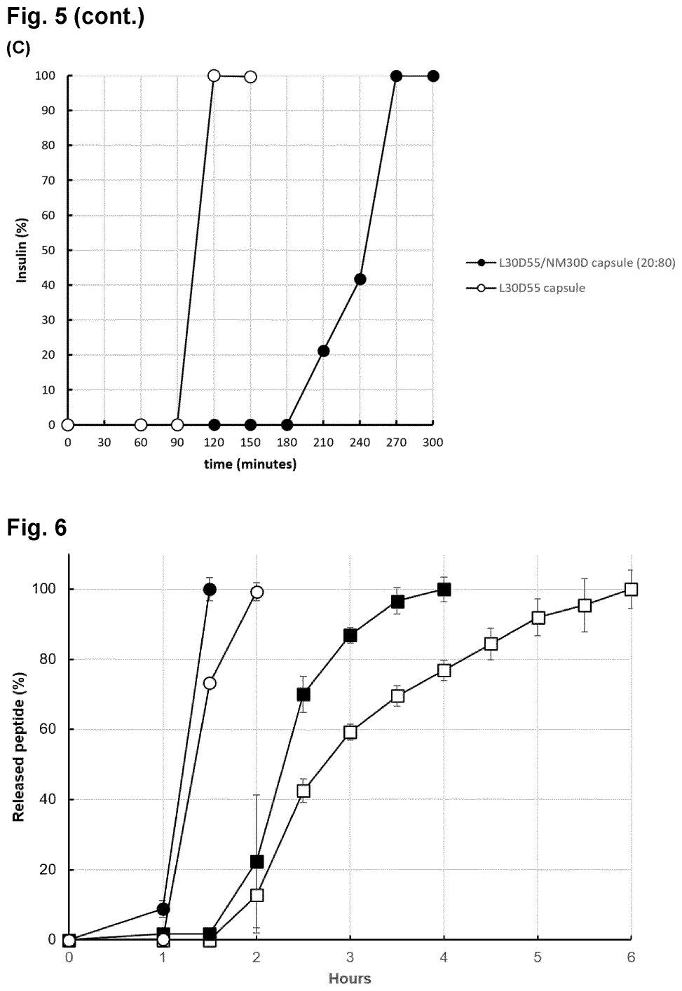

Tablets from Examples 24 and 25 were used for dissolution studies. Dissolution studies were performed with an Erweka DT light 126, using the basket method at 37° C. and a rotation speed of 75 rpm. A modified version of the United States Pharmacopeia (USP) method was used. The tablets were put into the baskets and the baskets were placed in open blue cap bottles, containing 100 ml of the corresponding dissolution media (SGF or SIF, as mentioned below). The blue cap bottles were in direct contact with water, ensuring a core temperature inside the blue cap bottles of 37° C. (temperature was confirmed prior to starting the test with an external thermometer). Baskets were first placed into simulated gastric fluid (SGF) according to USP for one hour, then for 5 hours in simulated intestinal fluid (SIF) according to USP with pH 7.4. Samples of 1 ml were withdrawn after 60 minutes and at further pre-determined time-points. 40 μl of the samples were injected into a HPLC system using a reversed phase gradient method (water/acetonitrile+0.1% trifluoroacetic acid; column: Kinetex C18, Phenomenex). Results from dissolution studies are shown in FIG. 6.

In conclusion, the above-reported in vivo studies (see Examples 20 to 23) with structurally different peptides, i.e.
a) Insulin (a polypeptide with disulfide bonds)
b) Leuprolide (a linear peptide)
c) Octreotide (a cyclic peptide)
d) Semaglutide (a fatty acid acylated peptide)
   showed improved oral bioavailability with sodium caprate tablets when the enteric coating comprised Eudragit NM30D in combination with a pH dependent Eudragit polymer such as Eudragit L30D-55 or Eudragit FS30D.

These results further confirm that the solid oral pharmaceutical compositions according to the present invention exhibit highly advantageous pharmacokinetic properties, including an advantageously improved oral bioavailability, as demonstrated in vivo using monkeys and pigs.

1. A solid oral pharmaceutical composition comprising:
(i) a core comprising a peptide or protein drug, and
(ii) a first coating, wherein the first coating comprises
(ii-1) a copolymer (A) in combination with
(ii-2) a copolymer (B) and/or a copolymer (C) and/or a copolymer (D);
wherein the copolymer (A) comprises:
(a) 20 to 90 mol-% ethyl acrylate repeating units, and
(b) 10 to 80 mol-% methyl methacrylate repeating units;
wherein the copolymer (B), if present, comprises:
(a) 25 to 75 mol-% methacrylic acid repeating units, and
(b) 25 to 75 mol-% ethyl acrylate repeating units;
wherein the copolymer (C), if present, comprises:
(a) 25 to 60 mol-% methacrylic acid repeating units, and
(b) 40 to 75 mol-% methyl methacrylate repeating units;
wherein the copolymer (D), if present, comprises:
(a) 5 to 20 mol-% methacrylic acid repeating units, and
(b) 20 to 40 mol-% methyl methacrylate repeating units, and
(c) 60 to 75 mol-% methyl acrylate repeating units.

2. The solid oral pharmaceutical composition according to claim 1, wherein the first coating comprises
(ii-1) a copolymer (A) in combination with
(ii-2) a copolymer (B) and/or a copolymer (D).

3. The solid oral pharmaceutical composition according to claim 1, wherein the copolymer (A) in the first coating comprises 60 to 75 mol-% ethyl acrylate repeating units, and 25 to 40 mol-% methyl methacrylate repeating units.

4. The solid oral pharmaceutical composition according to claim 1, wherein the copolymer (A) in the first coating comprises ethyl acrylate repeating units and methyl methacrylate repeating units in a molar ratio of 2:1.

5. The solid oral pharmaceutical composition according to claim 1, wherein the copolymer (A) in the first coating further comprises 0.5 to 20 mol-% 2-(trimethylammonio)ethyl methacrylate chloride repeating units.

6. The solid oral pharmaceutical composition according to claim 1, wherein the copolymer (B) in the first coating comprises 45 to 55 mol-% methacrylic acid repeating units, and 45 to 55 mol-% ethyl acrylate repeating units.

7. The solid oral pharmaceutical composition according to claim 1, wherein the copolymer (B) in the first coating comprises methacrylic acid repeating units and ethyl acrylate repeating units in a molar ratio of 1:1.

8. The solid oral pharmaceutical composition according to claim 1, wherein the copolymer (B) in the first coating consists of methacrylic acid repeating units and ethyl acrylate repeating units.

9. The solid oral pharmaceutical composition according to claim 1, wherein the first coating comprises the copolymer (A) and the copolymer (B), wherein the content of the copolymer (A) in the first coating is at least 25% (w/w) in relation to the total weight of the copolymer (A) and the copolymer (B) in the first coating.

10. The solid oral pharmaceutical composition according to claim 1, further comprising:
(iii) a second coating which is exterior to the first coating, wherein the second coating comprises a copolymer (C);
wherein the copolymer (C) comprises:
(a) 25 to 60 mol-% methacrylic acid repeating units, and
(b) 40 to 75 mol-% methyl methacrylate repeating units.

11. The solid oral pharmaceutical composition according to claim 1, wherein the peptide or protein drug has a molecular weight of equal to or less than about 300 kDa.

12. The solid oral pharmaceutical composition according to claim 1, wherein the peptide or protein drug is selected from insulin, an insulin analog, insulin lispro, insulin PEGlispro, insulin aspart, insulin glulisine, insulin glargine, insulin detemir, NPH insulin, insulin degludec, B29K(N(ε)hexadecanedioyl-γ-L-Glu) A14E B25H desB30 human insulin, B29K(N(ε)octadecanedioyl-γ-L-Glu-OEG-OEG) desB30 human insulin, B29K(N(ε)octadecanedioyl-γ-L-Glu) A14E B25H desB30 human insulin, B29K(N(ε)eicosanedioyl-γ-L-Glu) A14E B25H desB30 human insulin, B29K(N(ε)octadecanedioyl-γ-L-Glu-OEG-OEG) A14E B25H desB30 human insulin, B29K(N(ε)eicosanedioyl-γ-L-Glu-OEG-OEG) A14E B25H desB30 human insulin, B29K(N(ε)eicosanedioyl-γ-L-Glu-OEG-OEG) A14E B16H B25H desB30 human insulin, B29K(N(ε)hexadecanedioyl-γ-L-Glu) A14E B16H B25H desB30 human insulin, B29K(N(ε)eicosanedioyl-γ-L-Glu-OEG-OEG) A14E B16H B25H desB30 human insulin, B29K(N(ε)octadecanedioyl) A14E B25H desB30 human insulin, GLP-2, a GLP-2 analog, a GLP-2 agonist, teduglutide, elsiglutide, glucose-dependent insulinotropic polypeptide, elamipretide, a cyclotide, recombinant factor VIIa, eptacog alfa, amylin, an amylin analog, pramlintide, a somatostatin analog, octreotide, lanreotide, pasireotide, goserelin, buserelin, leptin, a leptin analog, metreleptin, peptide YY, a peptide YY analog, glatiramer, leuprolide, desmopressin, a desmopressin analog, a vasopressin receptor 2 agonist peptide, osteocalcin, an osteocalcin analog or derivative, human growth hormone, a human growth hormone analog, a long-acting human growth hormone, fibroblast growth factor 21, somapacitan, hGH-CTP, an antibody, a glycopeptide antibiotic, a glycosylated cyclic or polycyclic nonribosomal peptide antibiotic, vancomycin, teicoplanin, telavancin, bleomycin, ramoplanin, decaplanin, a cyclotide, bortezomib, cosyntropin, chorionic gonadotropin, menotropin, sermorelin, luteinizing-hormone-releasing hormone, somatropin, calcitonin, calcitonin-salmon, pentagastrin, oxytocin, nesiritide, anakinra, enfuvirtide, pegvisomant, dornase alfa, lepirudin, anidulafungin, eptifibatide, interferon alfacon-1, interferon alpha-2a, interferon alpha-2b, interferon beta-1a, interferon beta-1b, interferon gamma-1b, peginterferon alfa-2a, peginterferon alfa-2b, peginterferon beta-1a, fibrinolysin, vasopressin, aldesleukin, an epoetin, epoetin alfa, darbepoetin alfa, epoetin beta, epoetin delta, epoetin omega, epoetin zeta, epoetin theta, methoxy polyethylene glycol-epoetin beta, continuous erythropoietin receptor activator, pegylated epo, albupoetin, an epo-dimer analogue, epo-Fc, carbamylated EPO, synthetic erythropoese protein, the low molecular epo analogue PBI-1402, filgrastim, PEG-filgrastim, interleukin-11, cyclosporine, glucagon, urokinase, viomycin, thyrotropin-releasing hormone, leucine-enkephalin, methionine-enkephalin, substance P, adrenocorticotropic hormone, parathyroid hormone, a parathyroid hormone fragment, teriparatide, PTH(1-31), PTH(2-34), parathyroid hormone-related protein, abaloparatide, linaclotide, carfilzomib, icatibant, ecallantide, cilengitide, a prostaglandin F2a receptor modulator, PDC31, abciximab, ranibizumab, alefacept, romiplostim, anakinra, abatacept, belatacept, and pharmaceutically acceptable salts thereof.

13. The solid oral pharmaceutical composition according to claim 1, wherein the peptide or protein drug is selected from GLP-2, a GLP-2 agonist, a GLP-2 analog, teduglutide, elsiglutide, insulin, human insulin, an insulin analog, insulin lispro, insulin PEGlispro, A14E B25H B29K(N(eps)octadecanedioyl-gGlu-OEG-OEG) desB30 human insulin, insulin aspart, insulin glulisine, insulin glargine, insulin detemir, NPH insulin, insulin degludec, an antibody, a somatostatin analog, octreotide, lanreotide, pasireotide, desmopressin, a desmopressin analog, a vasopressin receptor 2 agonist peptide, a parathyroid hormone fragment, teriparatide, PTH(1-31), and PTH(2-34).

14. The solid oral pharmaceutical composition according to claim 1, wherein the peptide or protein drug is selected from an anti-obesity peptide, a neuropeptide Y receptor (NPY) agonist peptide, a NPY receptor Y1 agonist peptide, a NPY receptor Y2 agonist peptide, a NPY receptor Y4 agonist peptide, a NPY receptor Y5 agonist peptide, a pancreatic polypeptide receptor agonist peptide, neuropeptide Y, peptide YY, PYY₃₋₃₆, a PYY analog or derivative, a long acting fatty acid acylated PYY analog, a neuropeptide FF receptor type 2 (NPFF2R) agonist, a G-protein coupled receptor 10 (GPR10) agonist, a fatty acid acylated dual GPR10-NPFF2R co-agonist, pancreatic polypeptide, prolactin releasing peptide (PrRP), a long acting PrRP31 analog, a C18 lipidated PrRP31 analog, GT001, PYY-1875, a leptin receptor agonist peptide, leptin, a leptin analog or derivative, a long acting fatty acid acylated leptin analog, a ghrelin receptor antagonist peptide, amylin, an amylin analog or derivative, a long acting fatty acid acylated amylin analog, pramlintide, cagrilintide, ZP8396, a gastric inhibitory polypeptide (GIP) receptor agonist peptide, a gastric inhibitory polypeptide (GIP) analog or derivative, a long-acting acylated GIP analog, a GIP agonist, a dual- or tri-agonist GIP peptide, ZP6590, a glucagon receptor agonist peptide, glucagon, a glucagon analog or derivative, a long-acting acylated glucagon analog, a GLP-2 receptor agonist peptide, GLP-2, a GLP-2 analog or derivative, a long-acting acylated GLP-2 analog, teduglutide, glepaglutide, apraglutide, dapiglutide, elsiglutide, forigerimod, EA-230, difelikefalin acetate, an agonist of κ-opioid receptor (KOR), aviptadil, a zonulin antagonist, larazotide, brimapitide, a Small Integrin-Binding Ligand N-linked Glycoprotein (SIBLING), TPX-100, ZP9830, a Kv1.3 ion channel blocker, ZP10000, an α4β7 integrin inhibitor, a pellino-1 protein-protein interaction inhibitor peptide, BBT-401, an alpha-4-beta-7 (α4β7) integrin antagonist, PN-943, an interleukin (IL) receptor targeted peptide, an IL-23 receptor targeted peptide, PN-235, PN-232, an IL-23 receptor antagonist, JNJ-77242113, a nanobody, an anti-IL13/OX40L nanobody, an anti-IL-6R nanobody, vobarilizumab, a single-domain antibody, caplacizumab, dolcanatide, a WNT5A-mimicking peptide, Foxy-5, a thrombospondin-1 (Tsp-1) expression inducing peptide, cyclic pentapeptide VT1021, a CXCR4 antagonist, balixafortide, an anticytokine peptide, BNZ132-1-40, a FK506-binding protein like (FKBPL) peptide, ALM-201, fexapotide triflutate, tyroserleutide, a luteinizing hormone-releasing hormone (LHRH) antagonist acting on gonadotropin-releasing hormone (GnRH) receptors, ozarelix, an LHRH natural ligand derivative, EP-100, a somatostatin receptor agonist, HTL0030310, vosoritide, a relaxin receptor modulator peptide, relaxin, a relaxin analog or derivative, a long-acting acylated relaxin analog, serelaxin, davunetide, zilucoplan, and alirinetide.

15. The solid oral pharmaceutical composition according to claim 1, wherein the solid oral pharmaceutical composition is an oral dosage form.

16. The solid oral pharmaceutical composition according to claim 15, wherein the solid oral pharmaceutical composition is in the form of a capsule or a tablet.

17. The solid oral pharmaceutical composition according to claim 15, wherein the core is in the form of a multiparticulate, a granulate or pellets.

18. The solid oral pharmaceutical composition according to claim 1, wherein the first coating comprises the copolymer (A) and the copolymer (B), wherein the content of the copolymer (A) in the first coating is at least 50% (w/w) in relation to the total weight of the copolymer (A) and the copolymer (B) in the first coating.

19. The solid oral pharmaceutical composition according to claim 1, wherein the first coating comprises the copolymer (A) and the copolymer (B), wherein the content of the copolymer (A) in the first coating is at least 80% (w/w) in relation to the total weight of the copolymer (A) and the copolymer (B) in the first coating.

20. The solid oral pharmaceutical composition according to claim 1, wherein said peptide or protein drug is not a GLP-1 receptor agonist.


SEQUENCE LISTING (1 sequence)

SEQ ID NO: 1 - protein, 33 aa, synthetic construct
  VARIANT 1: note = L-His, imidazopropionyl, alpha-hydroxy-His, D-His, deamino-His, 2-amino-His, beta-hydroxy-His, homo-His, N-alpha-acetyl-His, N-alpha-formyl-His, alpha-fluoromethyl-His, alpha-methyl-His, 3-pyridyl-Ala, 2-pyridyl-Ala, or 4-pyridyl-Ala
  VARIANT 2: note = A, G, V, L, I, T, S, K, alpha-aminoisobutyric acid, (1-aminocyclopropyl)carboxylic acid, (1-aminocyclobutyl)carboxylic acid, (1-aminocyclopentyl)carboxylic acid, (1-aminocyclohexyl)carboxylic acidcarboxylic acid, or (1-aminocycloheptyl)carboxylic acid
  VARIANT 6: note = K or F
  VARIANT 10: note = V or L
  VARIANT 12: note = S, R, N, Q, or E
  VARIANT 13: note = Y or Q
  VARIANT 14: note = L, K, or M
  VARIANT 16: note = G, E, K, or alpha-aminoisobutyric acid
  VARIANT 17: note = Q, E, or R
  VARIANT 18: note = A or K
  VARIANT 19: note = A or V
  VARIANT 20: note = V, H, K or R
  VARIANT 24: note = A, E, or R
  VARIANT 25: note = W or H
  VARIANT 28: note = E, N, G, Q, or R
  VARIANT 29: note = G, alpha-aminoisobutyric acid, or absent
  VARIANT 30: note = R, G, K, or absent
  VARIANT 31: note = G, A, E, P, K, R, or absent
  VARIANT 32: note = S, G, A, E, Q, P, R, or absent
  VARIANT 33: note = G or absent
  source 1..33: note = Based on human GLP-1
XXEGTXTSDXSXXXEXXXXXKFIXXLVXXXXXX